(19) United States
(12) Patent Application Publication
(10) Pub. No.: US 2002/0044949 A1
(43) Pub. Date: Apr. 18, 2002

(54) 76 KDA HELICOBACTER POLYPEPTIDES AND CORRESPONDING POLYNUCLEOTIDE MOLECULES

(76) Inventors: HAROLD KLEANTHOUS, NEWTONVILLE, MA (US); LING LISSOLO, MARCY L'EBOILE (FR); JEAN-FRANCOIS TOMB, BALTIMORE, MD (US); CHARLES MILLER, MEDFORD, MA (US); AMAL AL-GARAWI, BOSTON, MA (US)

Correspondence Address: PAUL T CLARK, CLARK AND ELBING, 176 FEDERAL STREET, BOSTON, MA, 021102223

(21) Appl. No.: 08/834,666

(22) Filed: Apr. 1, 1997

(51) Int. Cl. A61K 39/02

(52) U.S. Cl. 424/235.1

(57) ABSTRACT

The invention provides 76 kDa Helicobacter polypeptides, which can be used in vaccination methods for preventing or treating Helicobacter infection, and polynucleotides that encode these polypeptides.

The invention relates to a family of 76 kDa Helicobacter antigens and corresponding polynucleotide molecules that can be used in methods to prevent or treat Helicobacter infection in mammals, such as humans.

### BACKGROUND OF THE INVENTION

Helicobacter is a genus of spiral, gram-negative bacteria that colonize the gastrointestinal tracts of mammals. Several species colonize the stomach, most notably H. pylori, H. heilmanii, H. felis, and H. mustelae. Although H. pylori is the species most commonly associated with human infection, H. heilmanii and H. felis have also been isolated from humans, but at lower frequencies than H. pylori.

Helicobacter infects over 50% of adult populations in developed countries and nearly 100% in developing countries and some Pacific rim countries, making it one of the most prevalent infections worldwide.

Helicobacter is routinely recovered from gastric biopsies of humans with histological evidence of gastritis and peptic ulceration. Indeed, H. pylori is now recognized as an important pathogen of humans, in that the chronic gastritis it causes is a risk factor for the development of peptic ulcer diseases and gastric carcinoma. It is thus highly desirable to develop safe and effective vaccines for preventing and treating Helicobacter infection.

A number of Helicobacter antigens have been characterized or isolated. These include urease, which is composed of two structural subunits of approximately 30 and 67 kDa (Hu et al., Infect. Immun. 58:992, 1990; Dunn et al., J. Biol. Chem. 265:9464, 1990; Evans et al., Microbial Pathogenesis 10:15, 1991; Labigne et al., J. Bact., 173:1920, 1991); the 87 kDa vacuolar cytotoxin (VacA) (Cover et al., J. Biol. Chem. 267:10570, 1992; Phadnis et al., Infect. Immun. 62:1557, 1994; WO 93/18150); a 128 kDa immunodominant antigen associated with the cytotoxin (CagA, also called TagA; WO 93/18150; U.S. Pat. No. 5,403,924); 13 and 58 kDa heat shock proteins HspA and HspB (Suerbaum et al., Mol. Microbiol. 14:959, 1994; WO 93/18150); a 54 kDa catalase (Hazell et al., J. Gen. Microbiol.137:57, 1991); a 15 kDa histidine-rich protein (Hpn) (Gilbert et al., Infect. Immun. 63:2682, 1995); a 20 kDa membrane-associated lipoprotein (Kostrcynska et al., J. Bact. 176:5938, 1994); a 30 kDa outer membrane protein (Bölin et al., J. Clin. Microbiol. 33:381, 1995); a lactoferrin receptor (FR 2,724,936); and several porins, designated HopA, HopB, HopC, HopD, and HopE, which have molecular weights of 48-67 kDa (Exner et al., Infect. Immun. 63:1567, 1995; Doig et al., J. Bact. 177:5447, 1995). Some of these proteins have been proposed as potential vaccine antigens. In particular, urease is believed to be a vaccine candidate (WO 94/9823; WO 95/22987; WO 95/3824; Michetti et al., Gastroenterology 107:1002, 1994). Nevertheless, it is thought that several antigens may ultimately be necessary in a vaccine.

### SUMMARY OF THE INVENTION

The invention provides polynucleotide molecules that encode a family of 76 kDa Helicobacter polypeptides, designated GHPO 386, GHPO 789, GHPO 1516, GHPO 1197, GHPO 1180, GHPO 896, GHPO 711, GHPO 190, GHPO 185,

GHPO 1417, and GHPO 1414, which can be used, e.g., in methods to prevent, treat, or diagnose Helicobacter infection. The polypeptides include those having the amino acid sequences shown in SEQ ID NOs:2-22 (even numbers). Those skilled in the art will understand that the invention also includes polynucleotide molecules that encode mutants and derivatives of these polypeptides, which can result from the addition, deletion, or substitution of non-essential amino acids, as is described further below.

In addition to the polynucleotide molecules described above, the invention includes the corresponding polypeptides (i.e., polypeptides encoded by the polynucleotide molecules of the invention, or fragments thereof), and monospecific antibodies that specifically bind to these polypeptides.

The present invention has many applications and includes expression cassettes, vectors, and cells transformed or transfected with the polynucleotides of the invention. Accordingly, the present invention provides (i) methods for producing polypeptides of the invention in recombinant host systems and related expression cassettes, vectors, and transformed or transfected cells; (ii) live vaccine vectors, such as pox virus, Salmonella typhimurium, and Vibrio cholerae vectors, that contain polynucleotides of the invention (such vaccine vectors being useful in, e.g., methods for preventing or treating Helicobacter infection) in combination with a diluent or carrier, and related pharmaceutical compositions and associated therapeutic and/or prophylactic methods; (iii) therapeutic and/or prophylactic methods involving administration of polynucleotide molecules, either in a naked form or formulated with a delivery vehicle, polypeptides or mixtures of polypeptides, or monospecific antibodies of the invention, and related pharmaceutical compositions; (iv) methods for detecting the presence of Helicobacter in biological samples, which can involve the use of polynucleotide molecules, monospecific antibodies, or polypeptides of the invention; and (v) methods for purifying polypeptides of the invention by antibody-based affinity chromatography.

### BRIEF DESCRIPTION OF THE DRAWINGS

FIG. 1 is an alignment of the predicted amino acid sequences of GHPO 386 (SEQ ID NO:2), GHPO 789 (SEQ ID NO:4), and GHPO 1516 (SEQ ID NO:6), as well as a consensus sequence for the 76 kDa protein family.

FIG. 2 is an alignment of the predicted amino acid sequences of GHPO 1197 (SEQ ID NO:8), GHPO 1180 (SEQ ID NO:10), GHPO 896 (SEQ ID NO:12), GHPO 711 (SEQ ID NO:14), GHPO 190 (SEQ ID NO:16), GHPO 185 (SEQ ID NO:18), GHPO 1417 (SEQ ID NO:20), and GHPO 1414 (SEQ ID NO:22), as well as a consensus sequence for the 76 kDa protein family.

### DETAILED DESCRIPTION

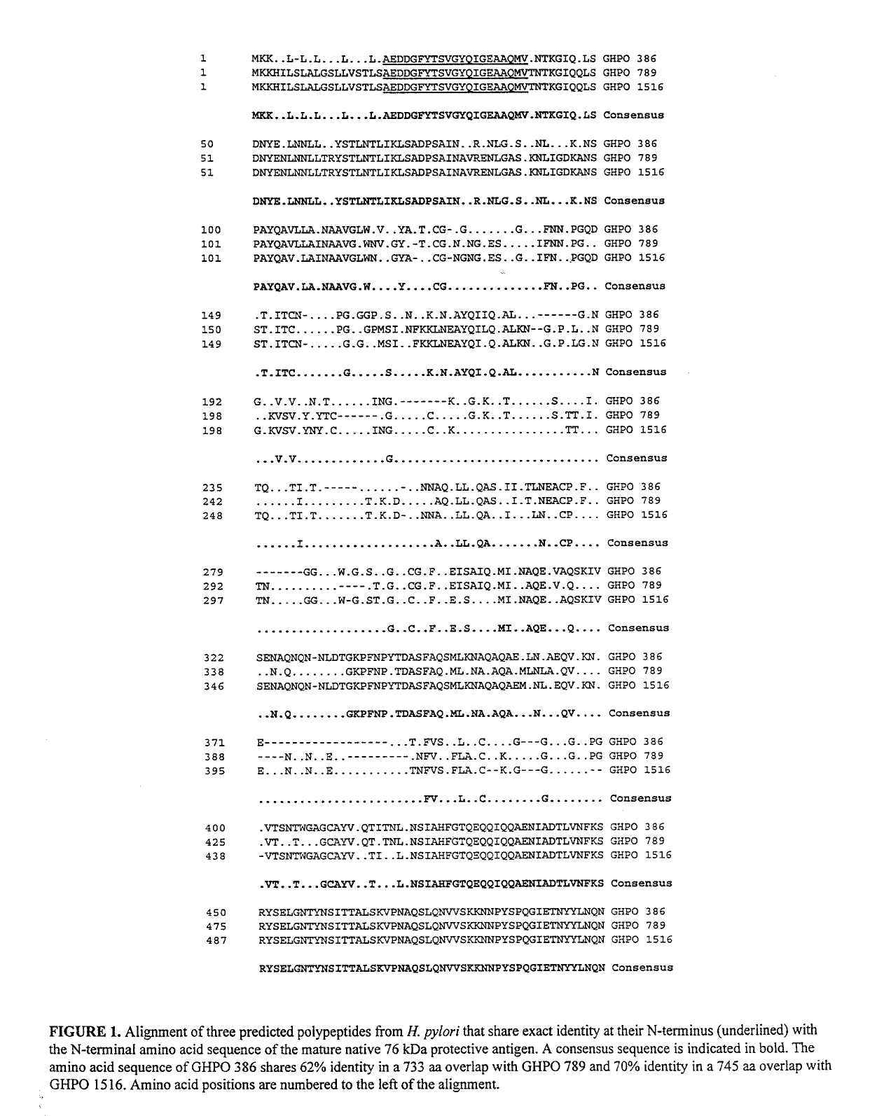

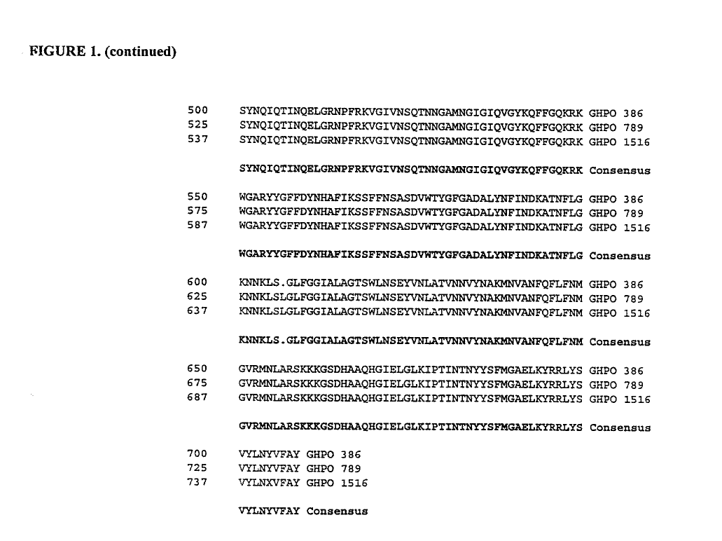

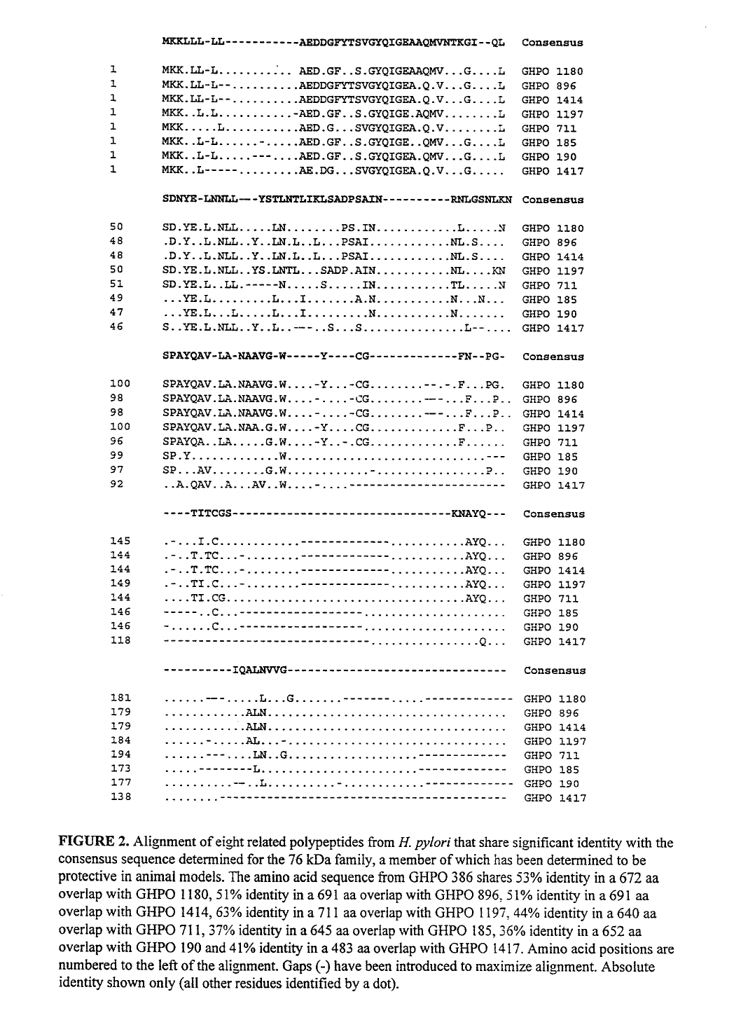

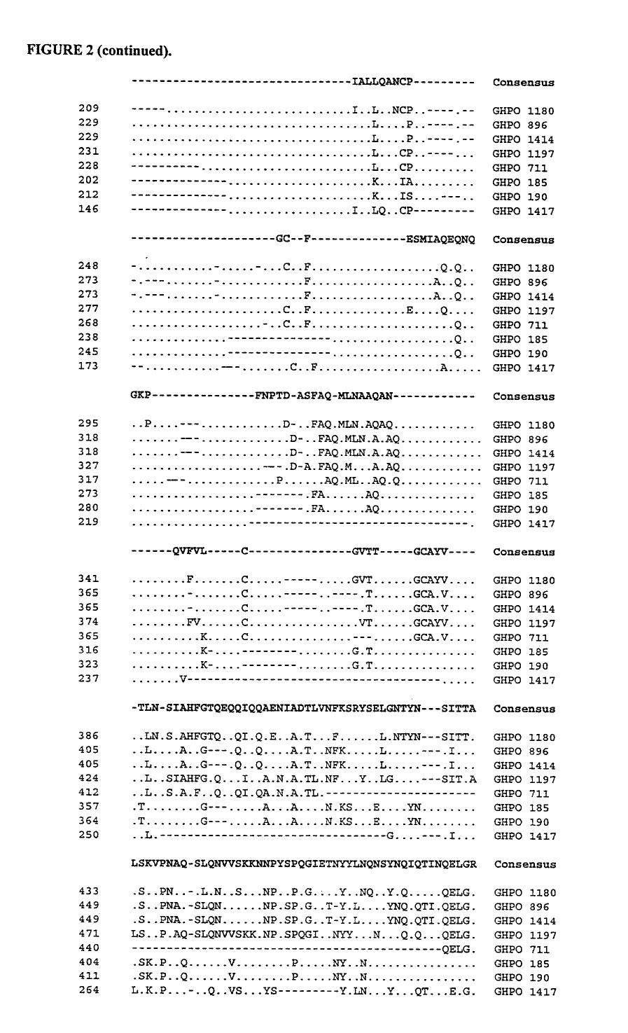

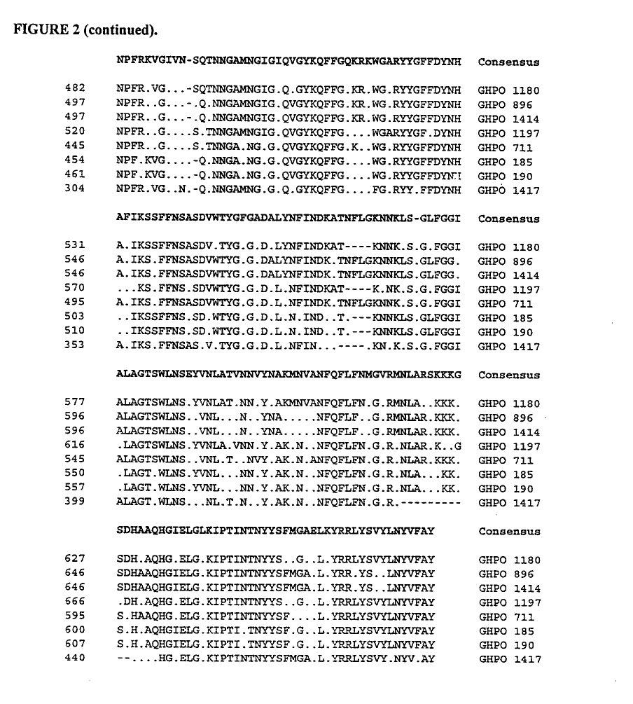

Open reading frames (ORFs) encoding a family of new, full length, membrane-associated 76 kDa polypeptides, designated GHPO 386, GHPO 789, GHPO 1516, GHPO 1197, GHPO 1180, GHPO 896, GHPO 711, GHPO 190, GHPO 185, GHPO 1417, and GHPO 1414, have been identified in the H. pylori genome. The amino acid sequences of these proteins are aligned in FIGS. 1 and 2. These polypeptides can be used, for example, in vaccination methods for preventing or treating Helicobacter infection. The polypeptides of the invention are secreted polypeptides that can be produced in their mature forms (i.e., as polypeptides that have been exported through class II or class III secretion pathways) or as precursors that include a signal peptide, which can be removed in the course of excretion/secretion by cleavage at the N-terminal end of the mature form. (The cleavage site is located at the C-terminal end of the signal peptide, adjacent to the mature form.) The cleavage site for the polypeptides of the invention and, thus, the first amino acid of the mature polypeptides, was putatively determined.

According to a first aspect of the invention, there are provided isolated polynucleotides that encode the precursor and mature forms of Helicobacter GHPO 386, GHPO 789, GHPO 1516, GHPO 1197, GHPO 1180, GHPO 896, GHPO 711, GHPO 190, GHPO 185, GHPO 1417, and GHPO 1414.

An isolated polynucleotide of the invention encodes:

(i) a polypeptide having an amino acid sequence that is homologous to a Helicobacter amino acid sequence of a polypeptide associated with the Helicobacter membrane, the Helicobacter amino acid sequence being selected from the group consisting of the amino acid sequences shown:

in SEQ ID NO:2, beginning with an amino acid in any one of positions −19 to 5, preferably in position −19 or position 1, and ending with an amino acid in position 689 (GHPO 386);

in SEQ ID NO:4, beginning with an amino acid in any one of positions −20 to 5, preferably in position −20 or position 1, and ending with an amino acid in position 713 (GHPO 789);

in SEQ ID NO:6, beginning with an amino acid in any one of positions −20 to 5, preferably in position −20 or position 1, and ending with an amino acid in position 725 (GHPO 1516);

in SEQ ID NO:8, beginning with an amino acid in any one of positions −20 to 5, preferably in position −20 or position 1, and ending with an amino acid in position 691 (GHPO 1197);

in SEQ ID NO:10, beginning with an amino acid in any one of positions −20 to 5, preferably in position −20 or position 1, and ending with an amino acid in position 652 (GHPO 1180);

in SEQ ID NO:12, beginning with an amino acid in any one of positions −18 to 5, preferably in position −18 or position 1, and ending with an amino acid in position 673 (GHPO 896);

in SEQ ID NO:14, beginning with an amino acid in any one of positions −21 to 5, preferably in position −21 or position 1, and ending with an amino acid in position 619 (GHPO 711);

in SEQ ID NO:16, beginning with an amino acid in any one of positions −17 to 5, preferably in position −17 or position 1, and ending with an amino acid in position 635 (GHPO 190);

in SEQ ID NO:18, beginning with an amino acid in any one of positions −19 to 5, preferably in position −19 or position 1, and ending with an amino acid in position 626 (GHPO 185);

in SEQ ID NO:20, beginning with an amino acid in any one of positions −16 to 5, preferably in position −16 or position 1, and ending with an amino acid in position 467 (GHPO 1417); and

in SEQ ID NO:22, beginning with an amino acid in any one of positions −18 to 5, preferably in position −18 or position 1, and ending with an amino acid in position 673 (GHPO 1414); or

(ii) a derivative of the polypeptide.

The term “isolated polynucleotide” is defined as a polynucleotide that is removed from the environment in which it naturally occurs. For example, a naturally-occurring DNA molecule present in the genome of a living bacteria or as part of a gene bank is not isolated, but the same molecule, separated from the remaining part of the bacterial genome, as a result of, e.g., a cloning event (amplification), is “isolated.” Typically, an isolated DNA molecule is free from DNA regions (e.g., coding regions) with which it is immediately contiguous, at the 5′ or 3′ ends, in the naturally occurring genome. Such isolated polynucleotides can be part of a vector or a composition and still be isolated, as such a vector or composition is not part of its natural environment.

A polynucleotide of the invention can consist of RNA or DNA (e.g., cDNA, genomic DNA, or synthetic DNA), or modifications or combinations of RNA or DNA. The polynucleotide can be double-stranded or single-stranded and, if single-stranded, can be the coding (sense) strand or the non-coding (anti-sense) strand. The sequences that encode polypeptides of the invention, as shown in SEQ ID NOs:2-22 (even numbers), can be (a) the coding sequence as shown in SEQ ID NOs:1-21 (odd numbers); (b) a ribonucleotide sequence derived by transcription of (a); or (c) a different coding sequence that, as a result of the redundancy or degeneracy of the genetic code, encodes the same polypeptides as the polynucleotide molecules having the sequences illustrated in SEQ ID NOs:1-21 (odd numbers). The polypeptides of the invention can be ones that are naturally secreted or excreted by, e.g., H. felis, H. mustelae, H. heilmanii, or H. pylori.

By “polypeptide” or “protein” is meant any chain of amino acids, regardless of length or post-translational modification (e.g., glycosylation or phosphorylation). Both terms are used interchangeably in the present application.

By “homologous amino acid sequence” is meant an amino acid sequence that differs from an amino acid sequence shown in any of SEQ ID NOs:2-22 (even numbers), or an amino acid sequence encoded by the nucleotide sequence of any of SEQ ID NOs:1-21 (odd numbers), by one or more non-conservative amino acid substitutions, deletions, or additions located at positions at which they do not destroy the specific antigenicity of the polypeptide. Preferably, such a sequence is at least 75%, more preferably at least 80%, and most preferably at least 90% identical to an amino acid sequence shown in any of SEQ ID NOs:2-22 (even numbers).

Homologous amino acid sequences include sequences that are identical or substantially identical to an amino acid sequence as shown in any of SEQ ID NOs:2-22 (even numbers). By “amino acid sequence that is substantially identical” is meant a sequence that is at least 90%, preferably at least 95%, more preferably at least 97%, and most preferably at least 99% identical to an amino acid sequence of reference and that differs from the sequence of reference, if at all, by a majority of conservative amino acid substitutions.

Conservative amino acid substitutions typically include substitutions among amino acids of the same class. These classes include, for example, amino acids having uncharged polar side chains, such as asparagine, glutamine, serine, threonine, and tyrosine; amino acids having basic side chains, such as lysine, arginine, and histidine; amino acids having acidic side chains, such as aspartic acid and glutamic acid; and amino acids having nonpolar side chains, such as glycine, alanine, valine, leucine, isoleucine, proline, phenylalanine, methionine, tryptophan, and cysteine.

Homology can be measured using sequence analysis software (e.g., Sequence Analysis Software Package of the Genetics Computer Group, University of Wisconsin Biotechnology Center, 1710 University Avenue, Madison, Wis. 53705). Similar amino acid sequences are aligned to obtain the maximum degree of homology (i.e., identity). To this end, it may be necessary to artificially introduce gaps into the sequence. Once the optimal alignment has been set up, the degree of homology (i.e., identity) is established by recording all of the positions in which the amino acids of both sequences are identical, relative to the total number of positions.

Homologous polynucleotide sequences are defined in a similar way. Preferably, a homologous sequence is one that is at least 45%, more preferably at least 60%, and most preferably at least 85% identical to a coding sequence of any of SEQ ID NOs:1-21 (odd numbers).

Polypeptides having a sequence homologous to one of the sequences shown in SEQ ID NOs:2-22 (even numbers) include naturally-occurring allelic variants, as well as mutants or any other non-naturally occurring variants that are analogous in terms of antigenicity, to a polypeptide having a sequence as shown in SEQ ID NOs:2-22 (even numbers).

As is known in the art, an allelic variant is an alternate form of a polypeptide that is characterized as having a substitution, deletion, or addition of one or more amino acids that does not alter the biological function of the polypeptide. By “biological function” is meant a function of the polypeptide in the cells in which it naturally occurs, even if the function is not necessary for the growth or survival of the cells. For example, the biological function of a porin is to allow the entry into cells of compounds present in the extracellular medium. The biological function is distinct from the antigenic function. A polypeptide can have more than one biological function.

Allelic variants are very common in nature. For example, a bacterial species, e.g., H. pylori, is usually represented by a variety of strains that differ from each other by minor allelic variations. Indeed, a polypeptide that fulfills the same biological function in different strains can have an amino acid sequence that is not identical in each of the strains. Such an allelic variation can be equally reflected at the polynucleotide level.

Support for the use of allelic variants of polypeptide antigens comes from, e.g., studies of the Helicobacter urease antigen. The amino acid sequence of Helicobacter urease varies widely from species to species, yet cross-species protection occurs, indicating that the urease molecule, when used as an immunogen, is highly tolerant of amino acid variations. Even among different strains of the single species H. pylori, there are amino acid sequence variations.

For example, although the amino acid sequences of the UreA and UreB subunits of H. pylori and H. felis ureases differ from one another by 26.5% and 11.8%, respectively (Ferrero et al., Molecular Microbiology 9(2):323-333, 1993), it has been shown that H. pylori urease protects mice from H. felis infection (Michetti et al., Gastroenterology 107:1002, 1994). In addition, it has been shown that the individual structural subunits of urease, UreA and UreB, which contain distinct amino acid sequences, are both protective antigens against Helicobacter infection (Michetti et al., supra). Similarly, Cuenca et al. (Gastroenterology 110:1770, 1996) showed that therapeutic immunization of H. mustelae-infected ferrets with H. pylori urease was effective at eradicating H. mustelae infection. Further, several urease variants have been reported to be effective vaccine antigens, including, e.g., recombinant UreA+UreB apoenzyme expressed from pORV142 (UreA and UreB sequences derived from H. pylori strain CPM630; Lee et al., J. Infect. Dis.172:161, 1995); recombinant UreA+UreB apoenzyme expressed from pORV214 (UreA and UreB sequences differ from H. pylori strain CPM630 by one and two amino acid changes, respectively; Lee et al., supra, 1995); a UreA-glutathione-S-transferase fusion protein (UreA sequence from H. pylori strain ATCC 43504; Thomas et al., Acta Gastro-Enterologica Belgica 56:54, 1993); UreA+UreB holoenzyme purified from H. pylori strain NCTCl 1637 (Marchetti et al., Science 267:1655, 1995); a UreA-MBP fusion protein (UreA from H. pylori strain 85P; Ferrero et al., Infection and Immunity 62:4981, 1994); a UreB-MBP fusion protein (UreB from H. pylori strain 85P; Ferrero et al., supra); a UreA-MBP fusion protein (UreA from H. felis strain ATCC 49179; Ferrero et al., supra); a UreB-MBP fusion protein (UreB from H. felis strain ATCC 49179; Ferrero et al., supra); and a 37 kDa fragment of UreB containing amino acids 220-569 (Dore-Davin et al., “A 37 kD fragment of UreB is sufficient to confer protection against Helicobacter felis infection in mice”). Finally, Thomas et al. (supra) showed that oral immunization of mice with crude sonicates of H. pylori protected mice from subsequent challenge with H. felis.

Polynucleotides, e.g., DNA molecules, encoding allelic variants can easily be obtained by polymerase chain reaction (PCR) amplification of genomic bacterial DNA extracted by conventional methods. This involves the use of synthetic oligonucleotide primers matching sequences that are upstream and downstream of the 5′ and 3′ ends of the coding region. Suitable primers can be designed based on the nucleotide sequence information provided in SEQ ID NOs:1-21 (odd numbers). Typically, a primer consists of 10 to 40, preferably 15 to 25 nucleotides. It can also be advantageous to select primers containing C and G nucleotides in proportions sufficient to ensure efficient hybridization, e.g., an amount of C and G nucleotides of at least 40%, preferably 50%, of the total nucleotide amount. Those skilled in the art can readily design primers that can be used to isolate the polynucleotides of the invention from different Helicobacter strains.

As an example, primers useful for cloning a DNA molecule encoding a polypeptide having the amino acid sequence of unprocessed GHPO 386 (SEQ ID NO:2), including a signal peptide, are shown in SEQ ID NO:23 (matching at the 5′ end) and in SEQ ID NO:25 (matching at the 3′ end). Primers useful for cloning a DNA molecule encoding a polypeptide having the amino acid sequence of mature GHPO 386 (amino acids 1-689 of SEQ ID NO:2), lacking a signal peptide, are shown in SEQ ID NO:24 (matching at the 5′ end) and in SEQ ID NO:25 (matching at the 3′ end). Experimental conditions for carrying out PCR can readily be determined by one skilled in the art and illustrations of carrying out PCR are provided in Examples 3 and 4.

Thus, the first aspect of the invention includes:

(i) isolated DNA molecules that can be amplified and/or cloned by the polymerase chain reaction from a Helicobacter, e.g., H. pylori, genome using either:

a 5′ oligonucleotide primer having a sequence as shown in SEQ ID NO:23, and a 3′ oligonucleotide primer having a sequence as shown in SEQ ID NO:25 (unprocessed GHPO 386);

a 5′ oligonucleotide primer having a sequence as shown in SEQ ID NO:26, and a 3′ oligonucleotide primer having a sequence as shown in SEQ ID NO:28 (unprocessed GHPO 789);

a 5′ oligonucleotide primer having a sequence as shown in SEQ ID NO:29, and a 3′ oligonucleotide primer having a sequence as shown in SEQ ID NO:31 (unprocessed GHPO 1516);

a 5′ oligonucleotide primer having a sequence as shown in SEQ ID NO:32, and a 3′ oligonucleotide primer having a sequence as shown in SEQ ID NO:34 (unprocessed GHPO 1197);

a 5′ oligonucleotide primer having a sequence as shown in SEQ ID NO:35, and a 3′ oligonucleotide primer having a sequence as shown in SEQ ID NO:37 (unprocessed GHPO 1180);

a 5′ oligonucleotide primer having a sequence as shown in SEQ ID NO:38, and a 3′ oligonucleotide primer having a sequence as shown in SEQ ID NO:40 (unprocessed GHPO 896);

a 5′ oligonucleotide primer having a sequence as shown in SEQ ID NO:41, and a 3′ oligonucleotide primer having a sequence as shown in SEQ ID NO:43 (unprocessed GHPO 711);

a 5′ oligonucleotide primer having a sequence as shown in SEQ ID NO:44, and a 3′ oligonucleotide primer having a sequence as shown in SEQ ID NO:46 (unprocessed GHPO 190);

a 5′ oligonucleotide primer having a sequence as shown in SEQ ID NO:47, and a 3′ oligonucleotide primer having a sequence as shown in SEQ ID NO:49 (unprocessed GHPO 185);

a 5′ oligonucleotide primer having a sequence as shown in SEQ ID NO:50, and a 3′ oligonucleotide primer having a sequence as shown in SEQ ID NO:52 (unprocessed GHPO 1417); or

a 5′ oligonucleotide primer having a sequence as shown in SEQ ID NO:53, and a 3′ oligonucleotide primer having a sequence as shown in SEQ ID NO:55 (unprocessed GHPO 1414); and

(ii) isolated DNA molecules that can be amplified and/or cloned by the polymerase chain reaction from a Helicobacter, e.g., H. pylori, genome using either:

a 5′ oligonucleotide primer having a sequence as shown in SEQ ID NO:24, and a 3′ oligonucleotide primer having a sequence as shown in SEQ ID NO:25 (mature GHPO 386);

a 5′ oligonucleotide primer having a sequence as shown in SEQ ID NO:27, and a 3′ oligonucleotide primer having a sequence as shown in SEQ ID NO:28 (mature GHPO 789);

a 5′ oligonucleotide primer having a sequence as shown in SEQ ID NO:30, and a 3′ oligonucleotide primer having a sequence as shown in SEQ ID NO:31 (mature GHPO 1516);

a 5′ oligonucleotide primer having a sequence as shown in SEQ ID NO:33, and a 3′ oligonucleotide primer having a sequence as shown in SEQ ID NO:34 (mature GHPO 1197);

a 5′ oligonucleotide primer having a sequence as shown in SEQ ID NO:36, and a 3′ oligonucleotide primer having a sequence as shown in SEQ ID NO:37 (mature GHPO 1180);

a 5′ oligonucleotide primer having a sequence as shown in SEQ ID NO:39, and a 3′ oligonucleotide primer having a sequence as shown in SEQ ID NO:40 (mature GHPO 896);

a 5′ oligonucleotide primer having a sequence as shown in SEQ ID NO:42, and a 3′ oligonucleotide primer having a sequence as shown in SEQ ID NO:43 (mature GHPO 711);

a 5′ oligonucleotide primer having a sequence as shown in SEQ ID NO:45, and a 3′ oligonucleotide primer having a sequence as shown in SEQ ID NO:46 (mature GHPO 190);

a 5′ oligonucleotide primer having a sequence as shown in SEQ ID NO:48, and a 3′ oligonucleotide primer having a sequence as shown in SEQ ID NO:49 (mature GHPO 185);

a 5′ oligonucleotide primer having a sequence as shown in SEQ ID NO:51, and a 3′ oligonucleotide primer having a sequence as shown in SEQ ID NO:52 (mature GHPO 1417); or

a 5′ oligonucleotide primer having a sequence as shown in SEQ ID NO:54, and a 3′ oligonucleotide primer having a sequence as shown in SEQ ID NO:55 (mature GHPO 1414).

In the sequences of SEQ ID NOs:23-55, the letter “N” denotes a restriction endonuclease digestion site that contains, typically, 4 to 6 nucleotides. For example, the sequences 5′-GGATCC-3′ (BamHI) or 5′-CTCGAG-3′ (XhoI) can be used. Restriction sites can be selected by those skilled in the art so that the amplified DNA can be conveniently cloned into an appropriately digested vector, such as a plasmid.

Useful homologs that do not occur naturally can be designed using known methods for identifying regions of an antigen that are likely to be tolerant of amino acid sequence changes and/or deletions. For example, sequences of the antigen from different species can be compared to identify conserved sequences.

Polypeptide derivatives that are encoded by polynucleotides of the invention include, e.g., fragments, polypeptides having large internal deletions derived from full-length polypeptides, and fusion proteins. Polypeptide fragments of the invention can be derived from a polypeptide having a sequence homologous to the sequences of any of SEQ ID NOs:2-22 (even numbers), to the extent that the fragments retain the substantial antigenicity of the parent polypeptide (specific antigenicity). Polypeptide derivatives can also be constructed by large internal deletions that remove a substantial part of the parent polypeptide, while retaining specific antigenicity. Generally, polypeptide derivatives should be about at least 12 amino acids in length to maintain antigenicity. Advantageously, they can be at least 20 amino acids, preferably at least 50 amino acids, more preferably at least 75 amino acids, and most preferably at least 100 amino acids in length.

Useful polypeptide derivatives, e.g., polypeptide fragments, can be designed using computer-assisted analysis of amino acid sequences in order to identify sites in protein antigens having potential as surface-exposed, antigenic regions (Hughes et al., Infect. Immun. 60(9):3497, 1992). For example, the Laser Gene Program from DNA Star can be used to obtain hydrophilicity, antigenic index, and intensity index plots for the polypeptides of the invention. This program can also be used to obtain information about homologies of the polypeptides with known protein motifs. One skilled in the art can readily use the information provided in such plots to select peptide fragments for use as vaccine antigens. For example, fragments spanning regions of the plots in which the antigenic index is relatively high can be selected. One can also select fragments spanning regions in which both the antigenic index and the intensity plots are relatively high. Fragments containing conserved sequences, particularly hydrophilic conserved sequences, can also be selected.

Polypeptide fragments and polypeptides having large internal deletions can be used for revealing epitopes that are otherwise masked in the parent polypeptide and that may be of importance for inducing a protective T cell-dependent immune response. Deletions can also remove immunodominant regions of high variability among strains.

It is an accepted practice in the field of immunology to use fragments and variants of protein immunogens as vaccines, as all that is required to induce an immune response to a protein is a small (e.g., 8 to 10 amino acids) immunogenic region of the protein. This has been done for a number of vaccines against pathogens other than Helicobacter. For example, short synthetic peptides corresponding to surface-exposed antigens of pathogens such as murine mammary tumor virus (peptide containing 11 amino acids; Dion et al., Virology 179:474-477, 1990), Semliki Forest virus (peptide containing 16 amino acids; Snijders et al., J. Gen. Virol. 72:557-565, 1991), and canine parvovirus (2 overlapping peptides, each containing 15 amino acids; Langeveld et al., Vaccine 12(15): 1473-1480, 1994) have been shown to be effective vaccine antigens against their respective pathogens.

Polynucleotides encoding polypeptide fragments and polypeptides having large internal deletions can be constructed using standard methods (see, e.g., Ausubel et al., Current Protocols in Molecular Biology, John Wiley & Sons Inc., 1994), for example, by PCR, including inverse PCR, by restriction enzyme treatment of the cloned DNA molecules, or by the method of Kunkel et al. (Proc. Natl. Acad. Sci. USA 82:448, 1985; biological material available at Stratagene).

A polypeptide derivative can also be produced as a fusion polypeptide that contains a polypeptide or a polypeptide derivative of the invention fused, e.g., at the N- or C-terminal end, to any other polypeptide (hereinafter referred to as a peptide tail). Such a product can be easily obtained by translation of a genetic fusion, i.e., a hybrid gene. Vectors for expressing fusion polypeptides are commercially available, and include the pMal-c2 or pMal-p2 systems of New England Biolabs, in which the peptide tail is a maltose binding protein, the glutathione-S-transferase system of Pharmacia, or the His-Tag system available from Novagen. These and other expression systems provide convenient means for further purification of polypeptides and derivatives of the invention.

Another particular example of fusion polypeptides included in invention includes a polypeptide or polypeptide derivative of the invention fused to a polypeptide having adjuvant activity, such as, e.g., subunit B of either cholera toxin or E. coli heat-labile toxin. Several possibilities can be used for producing such fusion proteins. First, the polypeptide of the invention can be fused to the N-terminal end or, preferably, to the C-terminal end of the polypeptide having adjuvant activity. Second, a polypeptide fragment of the invention can be fused within the amino acid sequence of the polypeptide having adjuvant activity. Spacer sequences can also be included, if desired.

As stated above, the polynucleotides of the invention encode Helicobacter polypeptides in precursor or mature form. They can also encode hybrid precursors containing heterologous signal peptides, which can mature into polypeptides of the invention. By “heterologous signal peptide” is meant a signal peptide that is not found in the naturally-occurring precursor of a polypeptide of the invention.

A polynucleotide of the invention hybridizes, preferably under stringent conditions, to a polynucleotide having a sequence as shown in any of SEQ ID NOs:1-21 (odd numbers). Hybridization procedures are, e.g., described by Ausubel et al. (supra); Silhavy et al. (Experiments with Gene Fusions, Cold Spring Harbor Laboratory Press, Cold Spring Harbor, N.Y., 1984); and Davis et al. (A Manual for Genetic Engineering: Advanced Bacterial Genetics, Cold Spring Harbor Laboratory Press, Cold Spring Harbor, N.Y., 1980). Important parameters that can be considered for optimizing hybridization conditions are reflected in the following formula, which facilitates calculation of the melting temperature (Tm), which is the temperature above which two complementary DNA strands separate from one another (Casey et al., Nucl. Acid Res. 4:1539, 1977): Tm=81.5+0.5×(% G+C)+1.6 log(positive ion concentration)−0.6×(% formamide). Under appropriate stringency conditions, hybridization temperature (Th) is approximately 20 to 40° C., 20 to 25° C., or, preferably, 30 to 40° C. below the calculated Tm. Those skilled in the art will understand that optimal temperature and salt conditions can be readily determined empirically in preliminary experiments using conventional procedures. For example, stringent conditions can be achieved, both for pre-hybridizing and hybridizing incubations, (i) within 4-16 hours at 42° C., in 6× SSC containing 50% formamide or (ii) within 4-16 hours at 65° C. in an aqueous 6× SSC solution (1 M NaCl, 0.1 M sodium citrate (pH 7.0)). For polynucleotides containing 30 to 600 nucleotides, the above formula is used and then is corrected by subtracting (600/polynucleotide size in base pairs). Stringency conditions are defined by a Th that is 5 to 10° C. below Tm.

Hybridization conditions with oligonucleotides shorter than 20-30 bases do not precisely follow the rules set forth above. In such cases, the formula for calculating the Tm is as follows: Tm=4×(G+C)+2(A+T). For example, an 18 nucleotide fragment of 50% G+C would have an approximate Tm of 54° C.

A polynucleotide molecule of the invention, containing RNA, DNA, or modifications or combinations thereof, can have various applications. For example, a polynucleotide molecule can be used (i) in a process for producing the encoded polypeptide in a recombinant host system, (ii) in the construction of vaccine vectors such as poxviruses, which are further used in methods and compositions for preventing and/or treating Helicobacter infection, (iii) as a vaccine agent, in a naked form or formulated with a delivery vehicle and, (iv) in the construction of attenuated Helicobacter strains that can over-express a polynucleotide of the invention or express it in a non-toxic, mutated form.

According to a second aspect of the invention, there is therefore provided (i) an expression cassette containing a polynucleotide molecule of the invention placed under the control of elements (e.g., a promoter) required for expression; (ii) an expression vector containing an expression cassette of the invention; (iii) a procaryotic or eucaryotic cell transformed or transfected with an expression cassette and/or vector of the invention, as well as (iv) a process for producing a polypeptide or polypeptide derivative encoded by a polynucleotide of the invention, which involves culturing a procaryotic or eucaryotic cell transformed or transfected with an expression cassette and/or vector of the invention, under conditions that allow expression of the polynucleotide molecule of the invention and, recovering the encoded polypeptide or polypeptide derivative from the cell culture.

A recombinant expression system can be selected from procaryotic and eucaryotic hosts. Eucaryotic hosts include, for example, yeast cells (e.g., Saccharomyces cerevisiae or Pichia Pastoris), mammalian cells (e.g., COS 1, NIH3T3, or JEG3 cells), arthropods cells (e.g., Spodoptera frugiperda (SF9) cells), and plant cells. Preferably, a procaryotic host such as E. coli is used. Bacterial and eucaryotic cells are available from a number of different sources that are known to those skilled in the art, e.g., the American Type Culture Collection (ATCC; Rockville, Md.).

The choice of the expression cassette will depend on the host system selected, as well as the features desired for the expressed polypeptide. For example, it may be useful to produce a polypeptide of the invention in a particular lipidated form or any other form. Typically, an expression cassette includes a constitutive or inducible promoter that is functional in the selected host system; a ribosome binding site; a start codon (ATG); if necessary, a region encoding a signal peptide, e.g., a lipidation signal peptide; a polynucleotide molecule of the invention; a stop codon; and, optionally, a 3′ terminal region (translation and/or transcription terminator). The signal peptide-encoding region is adjacent to the polynucleotide of the invention and is placed in the proper reading frame. The signal peptide-encoding region can be homologous or heterologous to the polynucleotide molecule encoding the mature polypeptide and it can be specific to the secretion apparatus of the host used for expression. The open reading frame constituted by the polynucleotide molecule of the invention, alone or together with the signal peptide, is placed under the control of the promoter so that transcription and translation occur in the host system. Promoters and signal peptide-encoding regions are widely known and available to those skilled in the art and include, for example, the promoter of Salmonella typhimurium (and derivatives) that is inducible by arabinose (promoter araB) and is functional in Gram-negative bacteria such as E. coli (U.S. Pat. No. 5,028,530; Cagnon et al., Protein Engineering 4(7):843, 1991); the promoter of the bacteriophage T7 RNA polymerase gene, which is functional in a number of E. coli strains expressing T7 polymerase (U.S. Pat. No. 4,952,496); the OspA lipidation signal peptide; and RlpB lipidation signal peptide (Takase et al., J. Bact. 169:5692, 1987).

The expression cassette is typically part of an expression vector, which is selected for its ability to replicate in the chosen expression system. Expression vectors (e.g., plasmids or viral vectors) can be chosen from, for example, those described in Pouwels et al. (Cloning Vectors: A Laboratory Manual, 1985, Supp. 1987) and can purchased from various commercial sources. Methods for transforming or transfecting host cells with expression vectors are well known in the art and will depend on the host system selected, as described in Ausubel et al. (supra).

Upon expression, a recombinant polypeptide of the invention (or a polypeptide derivative) is produced and remains in the intracellular compartment, is secreted/excreted in the extracellular medium or in the periplasmic space, or is embedded in the cellular membrane. The polypeptide can then be recovered in a substantially purified form from the cell extract or from the supernatant after centrifugation of the cell culture. Typically, the recombinant polypeptide can be purified by antibody-based affinity purification or by any other method known to a person skilled in the art, such as by genetic fusion to a small affinity-binding domain. Antibody-based affinity purification methods are also available for purifying a polypeptide of the invention extracted from a Helicobacter strain. Antibodies useful for immunoaffinity purification of the polypeptides of the invention can be obtained using methods described below.

Polynucleotides of the invention can also be used in DNA vaccination methods, using either a viral or bacterial host as gene delivery vehicle (live vaccine vector) or administering the gene in a free form, e.g., inserted into a plasmid. Therapeutic or prophylactic efficacy of a polynucleotide of the invention can be evaluated as is described below.

Accordingly, in a third aspect of the invention, there is provided (i) a vaccine vector such as a poxvirus, containing a polynucleotide molecule of the invention placed under the control of elements required for expression; (ii) a composition of matter containing a vaccine vector of the invention, together with a diluent or carrier; (iii) a pharmaceutical composition containing a therapeutically or prophylactically effective amount of a vaccine vector of the invention; (iv) a method for inducing an immune response against Helicobacter in a mammal (e.g., a human; alternatively, the method can be used in veterinary applications for treating or preventing Helicobacter infection of animals, e.g., cats or birds), which involves administering to the mammal an immunogenically effective amount of a vaccine vector of the invention to elicit an immune response, e.g., a protective or therapeutic immune response to Helicobacter; and (v) a method for preventing and/or treating a Helicobacter (e.g., H. pylori, H. felis, H. mustelae, or H. heilmanii) infection, which involves administering a prophylactic or therapeutic amount of a vaccine vector of the invention to an individual in need. Additionally, the third aspect of the invention encompasses the use of a vaccine vector of the invention in the preparation of a medicament for preventing and/or treating Helicobacter infection.

A vaccine vector of the invention can express one or several polypeptides or derivatives of the invention, as well as at least one additional Helicobacter antigen such as a urease apoenzyme or a subunit, fragment, homolog, mutant, or derivative thereof. In addition, it can express a cytokine, such as interleukin-2 (IL-2) or interleukin-12 (IL-12), that enhances the immune response. Thus, a vaccine vector can include an additional polynucleotide molecules encoding, e.g., urease subunit A, B, or both, or a cytokine, placed under the control of elements required for expression in a mammalian cell.

Alternatively, a composition of the invention can include several vaccine vectors, each of which being capable of expressing a polypeptide or derivative of the invention. A composition can also contain a vaccine vector capable of expressing an additional Helicobacter antigen such as urease apoenzyme, a subunit, fragment, homolog, mutant, or derivative thereof, or a cytokine such as IL-2 or IL-12.

In vaccination methods for treating or preventing infection in a mammal, a vaccine vector of the invention can be administered by any conventional route in use in the vaccine field, for example, to a mucosal (e.g., ocular, intranasal, oral, gastric, pulmonary, intestinal, rectal, vaginal, or urinary tract) surface or via a parenteral (e.g., subcutaneous, intradermal, intramuscular, intravenous, or intraperitoneal) route. Preferred routes depend upon the choice of the vaccine vector. The administration can be achieved in a single dose or repeated at intervals. The appropriate dosage depends on various parameters that are understood by those skilled in the art, such as the nature of the vaccine vector itself, the route of administration, and the condition of the mammal to be vaccinated (e.g., the weight, age, and general health of the mammal).

Live vaccine vectors that can be used in the invention include viral vectors, such as adenoviruses and poxviruses, as well as bacterial vectors, e.g., Shigella, Salmonella, Vibrio cholerae, Lactobacillus, Bacille bilié de Calmette-Guérin (BCG), and Streptococcus. An example of an adenovirus vector, as well as a method for constructing an adenovirus vector capable of expressing a polynucleotide molecule of the invention, is described in U.S. Pat. No. 4,920,209. Poxvirus vectors that can be used in the invention include, e.g., vaccinia and canary pox viruses, which are described in U.S. Pat. No. 4,722,848 and U.S. Pat. No. 5,364,773, respectively (also see, e.g., Tartaglia et al., Virology 188:217, 1992, for a description of a vaccinia virus vector, and Taylor et al, Vaccine 13:539, 1995, for a description of a canary poxvirus vector). Poxvirus vectors capable of expressing a polynucleotide of the invention can be obtained by homologous recombination, as described in Kieny et al. (Nature 312:163, 1984) so that the polynucleotide of the invention is inserted in the viral genome under appropriate conditions for expression in mammalian cells. Generally, the dose of viral vector vaccine, for therapeutic or prophylactic use, can be from about 1×10⁴, to about 1×10¹¹, advantageously from about 1×10⁷ to about 1×10¹¹, or, preferably, from about 1×10⁷ to about 1×10⁹ plaque-forming units per kilogram. Preferably, viral vectors are administered parenterally, for example, in 3 doses that are 4 weeks apart. Those skilled in the art will recognize that it is preferable to avoid adding a chemical adjuvant to a composition containing a viral vector of the invention and thereby minimizing the immune response to the viral vector itself.

Non-toxicogenic Vibrio cholerae mutant strains that can be used in live oral vaccines are described by Mekalanos et al. (Nature 306:551, 1983) and in U.S. Pat. No. 4,882,278 (strain in which a substantial amount of the coding sequence of each of the two ctxA alleles has been deleted so that no functional cholerae toxin is produced); WO 92/11354 (strain in which the irgA locus is inactivated by mutation; this mutation can be combined in a single strain with ctxA mutations); and WO 94/1533 (deletion mutant lacking functional ctxA and attRS1 DNA sequences). These strains can be genetically engineered to express heterologous antigens, as described in WO 94/19482. An effective vaccine dose of a V. cholerae strain capable of expressing a polypeptide or polypeptide derivative encoded by a polynucleotide molecule of the invention can contain, e.g., about 1×10⁵ to about 1×10⁹, preferably about 1×10⁶ to about 1×10⁸ viable bacteria in an appropriate volume for the selected route of administration. Preferred routes of administration include all mucosal routes, but, most preferably, these vectors are administered intranasally or orally.

Attenuated Salmonella typhimurium strains, genetically engineered for recombinant expression of heterologous antigens, and their use as oral vaccines, are described by Nakayama et al. (Bio/Technology 6:693, 1988) and in WO 92/11361. Preferred routes of administration for these vectors include all mucosal routes. Most preferably, the vectors are administered intranasally or orally.

Others bacterial strains useful as vaccine vectors are described by High et al. (EMBO 11:1991, 1992) and Sizemore et al. (Science 270:299, 1995; Shigella flexneri); Medaglini et al. (Proc. Natl. Acad. Sci. USA 92:6868, 1995; (Streptococcus gordonii); Flynn (Cell. Mol. Biol. 40 (suppl. I):31, 1194), and in WO 88/6626, WO 90/0594, WO 91/13157, WO 92/1796, and WO 92/21376 (Bacille Calmette Guerin). In bacterial vectors, a polynucleotide of the invention can be inserted into the bacterial genome or it can remain in a free state, for example, carried on a plasmid.

An adjuvant can also be added to a composition containing a bacterial vector vaccine. A number of adjuvants that can be used are known to those skilled in the art. For example, preferred adjuvants can be selected from the list provided below.

According to a fourth aspect of the invention, there is also provided (i) a composition of matter containing a polynucleotide of the invention, together with a diluent or carrier; (ii) a pharmaceutical composition containing a therapeutically or prophylactically effective amount of a polynucleotide of the invention; (iii) a method for inducing an immune response against Helicobacter, in a mammal, by administering to the mammal an immunogenically effective amount of a polynucleotide of the invention to elicit an immune response, e.g., a protective immune response to Helicobacter; and (iv) a method for preventing and/or treating a Helicobacter (e.g., H. pylori, H. felis, H. mustelae, or H. heilmanii) infection, by administering a prophylactic or therapeutic amount of a polynucleotide of the invention to an individual in need of such treatment. Additionally, the fourth aspect of the invention encompasses the use of a polynucleotide of the invention in the preparation of a medicament for preventing and/or treating Helicobacter infection. The fourth aspect of the invention preferably includes the use of a polynucleotide molecule placed under conditions for expression in a mammalian cell, e.g., in a plasmid that is unable to replicate in mammalian cells and to substantially integrate into a mammalian genome.

Polynucleotides (for example, DNA or RNA molecules) of the invention can also be administered as such to a mammal as a vaccine. When a DNA molecule of the invention is used, it can be in the form of a plasmid that is unable to replicate in a mammalian cell and unable to integrate into the mammalian genome. Typically, a DNA molecule is placed under the control of a promoter suitable for expression in a mammalian cell. The promoter can function ubiquitously or tissue-specifically. Examples of non-tissue specific promoters include the early Cytomegalovirus (CMV) promoter (U.S. Pat. No. 4,168,062) and the Rous Sarcoma Virus promoter (Norton et al., Molec. Cell Biol. 5:281, 1985). The desmin promoter (Li et al., Gene 78:243, 1989; Li et al., J. Biol. Chem. 266:6562, 1991; Li et al., J. Biol. Chem. 268:10403, 1993) is tissue-specific and drives expression in muscle cells. More generally, useful promoters and vectors are described, e.g., in WO 94/21797 and by Hartikka et al. (Human Gene Therapy 7:1205, 1996).

For DNA/RNA vaccination, the polynucleotide of the invention can encode a precursor or a mature form of a polypeptide of the invention. When it encodes a precursor form, the precursor sequence can be homologous or heterologous. In the latter case, a eucaryotic leader sequence can be used, such as the leader sequence of the tissue-type plasminogen factor (tPA).

A composition of the invention can contain one or several polynucleotides of the invention. It can also contain at least one additional polynucleotide encoding another Helicobacter antigen, such as urease subunit A, B, or both, or a fragment, derivative, mutant, or analog thereof. A polynucleotide encoding a cytokine, such as interleukin-2 (IL-2) or interleukin-12 (IL-12), can also be added to the composition so that the immune response is enhanced. These additional polynucleotides are placed under appropriate control for expression. Advantageously, DNA molecules of the invention and/or additional DNA molecules to be included in the same composition are carried in the same plasmid.

Standard methods can be used in the preparation of therapeutic polynucleotides of the invention. For example, a polynucleotide can be used in a naked form, free of any delivery vehicles, such as anionic liposomes, cationic lipids, microparticles, e.g., gold microparticles, precipitating agents, e.g., calcium phosphate, or any other transfection-facilitating agent. In this case, the polynucleotide can be simply diluted in a physiologically acceptable solution, such as sterile saline or sterile buffered saline, with or without a carrier. When present, the carrier preferably is isotonic, hypotonic, or weakly hypertonic, and has a relatively low ionic strength, such as provided by a sucrose solution, e.g., a solution containing 20% sucrose.

Alternatively, a polynucleotide can be associated with agents that assist in cellular uptake. It can be, e.g., (i) complemented with a chemical agent that modifies cellular permeability, such as bupivacaine (see, e.g., WO 94/16737), (ii) encapsulated into liposomes, or (iii) associated with cationic lipids or silica, gold, or tungsten microparticles.

Anionic and neutral liposomes are well-known in the art (see, e.g., Liposomes: A Practical Approach, RPC New Ed, IRL Press, 1990, for a detailed description of methods for making liposomes) and are useful for delivering a large range of products, including polynucleotides.

Cationic lipids can also be used for gene delivery. Such lipids include, for example, Lipofectin™, which is also known as DOTMA (N-[1-(2,3-dioleyloxy)propyl]-N,N,N-trimethylammonium chloride), DOTAP (1,2-bis(oleyloxy)-3-(trimethylammonio)propane), DDAB (dimethyldioctadecylammonium bromide), DOGS (dioctadecylamidologlycyl spermine), and cholesterol derivatives. A description of these cationic lipids can be found in EP 187,702, WO 90/11092, U.S. Pat. No. 5,283,185, WO 91/15501, WO 95/26356, and U.S. Pat. No. 5,527,928. Cationic lipids for gene delivery are preferably used in association with a neutral lipid such as DOPE (dioleyl phosphatidylethanolamine; WO 90/11092). Other transfection-facilitating compounds can be added to a formulation containing cationic liposomes. A number of them are described in, e.g., WO 93/18759, WO 93/19768, WO 94/25608, and WO 95/2397. They include, e.g., spermine derivatives useful for facilitating the transport of DNA through the nuclear membrane (see, for example, WO 93/18759) and membrane-permeabilizing compounds such as GALA, Gramicidine S, and cationic bile salts (see, for example, WO 93/19768).

Gold or tungsten microparticles can also be used for gene delivery, as described in WO 91/359, WO 93/17706, and by Tang et al. (Nature 356:152, 1992). In this case, the microparticle-coated polynucleotides can be injected via intradermal or intraepidermal routes using a needleless injection device (“gene gun”), such as those described in U.S. Pat. No. 4,945,050, U.S. Pat. No. 5,015,580, and WO 94/24263.

The amount of DNA to be used in a vaccine recipient depends, e.g., on the strength of the promoter used in the DNA construct, the immunogenicity of the expressed gene product, the condition of the mammal intended for administration (e.g., the weight, age, and general health of the mammal), the mode of administration, and the type of formulation. In general, a therapeutically or prophylactically effective dose from about 1 μg to about 1 mg, preferably, from about 10 μg to about 800 μg, and, more preferably, from about 25 μg to about 250 μg, can be administered to human adults. The administration can be achieved in a single dose or repeated at intervals.

The route of administration can be any conventional route used in the vaccine field. As general guidance, a polynucleotide of the invention can be administered via a mucosal surface, e.g., an ocular, intranasal, pulmonary, oral, intestinal, rectal, vaginal, or urinary tract surface, or via a parenteral route, e.g., by an intravenous, subcutaneous, intraperitoneal, intradermal, intraepidermal, or intramuscular route. The choice of administration route will depend on, e.g., the formulation that is selected. A polynucleotide formulated in association with bupivacaine is advantageously administered into muscle. When a neutral or anionic liposome or a cationic lipid, such as DOTMA, is used, the formulation can be advantageously injected via intravenous, intranasal (for example, by aerosolization), intramuscular, intradermal, and subcutaneous routes. A polynucleotide in a naked form can advantageously be administered via the intramuscular, intradermal, or subcutaneous routes. Although not absolutely required, such a composition can also contain an adjuvant. A systemic adjuvant that does not require concomitant administration in order to exhibit an adjuvant effect is preferable.

The sequence information provided in the present application enables the design of specific nucleotide probes and primers that can be used in diagnostic methods. Accordingly, in a fifth aspect of the invention, there is provided a nucleotide probe or primer having a sequence found in, or derived by degeneracy of the genetic code from, a sequence shown in SEQ ID NOs:1-21 (odd numbers) or a complementary sequence thereof.

The term “probe” as used in the present application refers to DNA (preferably single stranded) or RNA molecules (or modifications or combinations thereof) that hybridize under the stringent conditions, as defined above, to polynucleotide molecules having sequences homologous to those shown in any of SEQ ID NOs:1-21 (odd numbers), or to a complementary or anti-sense sequence of any of SEQ ID NOs:1-21 (odd numbers). Generally, probes are significantly shorter than the full-length sequences shown in SEQ ID NOs:1-21 (odd numbers). For example, they can contain from about 5 to about 100, preferably from about 10 to about 80 nucleotides. In particular, probes have sequences that are at least 75%, preferably at least 85%, more preferably 95% homologous to a portion of a sequence as shown in SEQ ID NOs:1-21 (odd numbers) or a sequence complementary to such sequences.

Probes can contain modified bases, such as inosine, methyl-5-deoxycytidine, deoxyuridine, dimethylamino-5-deoxyuridine, or diamino-2, 6-purine. Sugar or phosphate residues can also be modified or substituted. For example, a deoxyribose residue can be replaced by a polyamide (Nielsen et al., Science 254:1497, 1991) and phosphate residues can be replaced by ester groups such as diphosphate, alkyl, arylphosphonate, and phosphorothioate esters. In addition, the 2′-hydroxyl group on ribonucleotides can be modified by addition of, e.g., alkyl groups.

Probes of the invention can be used in diagnostic tests, or as capture or detection probes. Such capture probes can be immobilized on solid supports, directly or indirectly, by covalent means or by passive adsorption. A detection probe can be labeled by a detectable label, for example a label selected from radioactive isotopes; enzymes, such as peroxidase and alkaline phosphatase; enzymes that are able to hydrolyze a chromogenic, fluorogenic, or luminescent substrate; compounds that are chromogenic, fluorogenic, or luminescent; nucleotide base analogs; and biotin.

Probes of the invention can be used in any conventional hybridization method, such as in dot blot methods (Maniatis et al., Molecular Cloning: A Laboratory Manual, Cold Spring Harbor Laboratory Press, Cold Spring Harbor, N.Y., 1982), Southern blot methods (Southern, J. Mol. Biol. 98:503, 1975), northern blot methods (identical to Southern blot to the exception that RNA is used as a target), or a sandwich method (Dunn et al., Cell 12:23, 1977). As is known in the art, the latter technique involves the use of a specific capture probe and a specific detection probe that have nucleotide sequences that are at least partially different from each other.

Primers used in the invention usually contain about 10 to 40 nucleotides and are used to initiate enzymatic polymerization of DNA in an amplification process (e.g., PCR), an elongation process, or a reverse transcription method. In a diagnostic method involving PCR, the primers can be labeled.

Thus, the invention also encompasses (i) a reagent containing a probe of the invention for detecting and/or identifying the presence of Helicobacter in a biological material; (ii) a method for detecting and/or identifying the presence of Helicobacter in a biological material, in which (a) a sample is recovered or derived from the biological material, (b) DNA or RNA is extracted from the material and denatured, and (c) the sample is exposed to a probe of the invention, for example, a capture probe, a detection probe, or both, under stringent hybridization conditions, so that hybridization is detected; and (iii) a method for detecting and/or identifying the presence of Helicobacter in a biological material, in which (a) a sample is recovered or derived from the biological material, (b) DNA is extracted therefrom, (c) the extracted DNA is contacted with at least one, or, preferably two, primers of the invention, and amplified by the polymerase chain reaction, and (d) an amplified DNA molecule is produced.

As mentioned above, polypeptides that can be produced by expression of the polynucleotides of the invention can be used as vaccine antigens.

Accordingly, a sixth aspect of the invention features a substantially purified polypeptide or polypeptide derivative having an amino acid sequence encoded by a polynucleotide of the invention.

A “substantially purified polypeptide” is defined as a polypeptide that is separated from the environment in which it naturally occurs and/or a polypeptide that is free of most of the other polypeptides that are present in the environment in which it was synthesized. The polypeptides of the invention can be purified from a natural source, such as a Helicobacter strain, or can be produced using recombinant methods.

Homologous polypeptides or polypeptide derivatives encoded by polynucleotides of the invention can be screened for specific antigenicity by testing cross-reactivity with an antiserum raised against a polypeptide having an amino acid sequence as shown in any of SEQ ID NOs:2-22 (even numbers). Briefly, a monospecific hyperimmune antiserum can be raised against a purified reference polypeptide as such or as a fusion polypeptide, for example, an expression product of MBP, GST, or His-tag systems, or a synthetic peptide predicted to be antigenic. The homologous polypeptide or derivative that is screened for specific antigenicity can be produced as such or as a fusion polypeptide. In the latter case, and if the antiserum is also raised against a fusion polypeptide, two different fusion systems are employed. Specific antigenicity can be determined using a number of methods, including Western blot (Towbin et al., Proc. Natl. Acad. Sci. USA 76:4350, 1979), dot blot, and ELISA methods, as described below.

In a Western blot assay, the product to be screened, either as a purified preparation or a total E. coli extract, is fractionated by SDS-PAGE, as described, for example, by Laemmli (Nature 227:680, 1970). After being transferred to a filter, such as a nitrocellulose membrane, the material is incubated with the monospecific hyperimmune antiserum, which is diluted in a range of dilutions from about 1:50 to about 1:5000, preferably from about 1:100 to about 1:500. Specific antigenicity is shown once a band corresponding to the product exhibits reactivity at any of the dilutions in the range.

In an ELISA assay, the product to be screened can be used as the coating antigen. A purified preparation is preferred, but a whole cell extract can also be used. Briefly, about 100 μL of a preparation of about 10 μg protein/mL is distributed into wells of a 96-well ELISA plate. The plate is incubated for about 2 hours at 37° C., then overnight at 4° C. The plate is washed with phosphate buffer saline (PBS) containing 0.05% Tween 20 (PBS/Tween buffer) and the wells are saturated with 250 μL PBS containing 1% bovine serum albumin (BSA), to prevent non-specific antibody binding. After 1 hour of incubation at 37° C., the plate is washed with PBS/Tween buffer. The antiserum is serially diluted in PBS/Tween buffer containing 0.5% BSA, and 100 μL dilutions are added to each well. The plate is incubated for 90 minutes at 37° C., washed, and evaluated using standard methods. For example, a goat anti-rabbit peroxidase conjugate can be added to the wells when the specific antibodies used were raised in rabbits. Incubation is carried out for about 90 minutes at 37° C. and the plate is washed. The reaction is developed with the appropriate substrate and the reaction is measured by colorimetry (absorbance measured spectrophotometrically). Under these experimental conditions, a positive reaction is shown once an O.D. value of 1.0 is detected with a dilution of at least about 1:50, preferably of at least about 1:500.

In a dot blot assay, a purified product is preferred, although a whole cell extract can be used. Briefly, a solution of the product at a concentration of about 100 μg/mL is serially diluted two-fold with 50 mM Tris-HCl (pH 7.5). One hundred μL of each dilution is applied to a filter, such as a 0.45 μm nitrocellulose membrane, set in a 96-well dot blot apparatus (Biorad). The buffer is removed by applying vacuum to the system. Wells are washed by addition of 50 mM Tris-HCl (pH 7.5) and the membrane is air-dried. The membrane is saturated in blocking buffer (50 mM Tris-HCl (pH 7.5), 0.15 M NaCl, 10 g/L skim milk) and incubated with an antiserum diluted from about 1:50 to about 1:5000, preferably about 1:500. The reaction is detected using standard methods. For example, a goat anti-rabbit peroxidase conjugate can be added to the wells when rabbit antibodies are used. Incubation is carried out for about 90 minutes at 37° C. and the blot is washed. The reaction is developed with the appropriate substrate and stopped. The reaction is then measured visually by the appearance of a colored spot, e.g., by colorimetry. Under these experimental conditions, a positive reaction is associated with detection of a colored spot for reactions carried out with a dilution of at least about 1:50, preferably, of at least about 1:500. Therapeutic or prophylactic efficacy of a polypeptide or polypeptide derivative of the invention can be evaluated as described below.

According to a seventh aspect of the invention, there is provided (i) a composition of matter containing a polypeptide of the invention together with a diluent or carrier; (ii) a pharmaceutical composition containing a therapeutically or prophylactically effective amount of a polypeptide of the invention; (iii) a method for inducing an immune response against Helicobacter in a mammal by administering to the mammal an immunogenically effective amount of a polypeptide of the invention to elicit an immune response, e.g., a protective immune response to Helicobacter; and (iv) a method for preventing and/or treating a Helicobacter (e.g., H. pylori, H. felis, H. mustelae, or H. heilmanii) infection, by administering a prophylactic or therapeutic amount of a polypeptide of the invention to an individual in need of such treatment. Additionally, this aspect of the invention includes the use of a polypeptide of the invention in the preparation of a medicament for preventing and/or treating Helicobacter infection.

The immunogenic compositions of the invention can be administered by any conventional route in use in the vaccine field, for example, to a mucosal (e.g., ocular, intranasal, pulmonary, oral, gastric, intestinal, rectal, vaginal, or urinary tract) surface or via a parenteral (e.g., subcutaneous, intradermal, intramuscular, intravenous, or intraperitoneal) route. The choice of the administration route depends upon a number of parameters, such as the adjuvant used. For example, if a mucosal adjuvant is used, the intranasal or oral route will be preferred, and if a lipid formulation or an aluminum compound is used, a parenteral route will be preferred. In the latter case, the subcutaneous or intramuscular route is most preferred. The choice of administration route can also depend upon the nature of the vaccine agent. For example, a polypeptide of the invention fused to CTB or to LTB will be best administered to a mucosal surface.

A composition of the invention can contain one or several polypeptides or derivatives of the invention. It can also contain at least one additional Helicobacter antigen, such as the urease apoenzyme, or a subunit, fragment, homolog, mutant, or derivative thereof.

For use in a composition of the invention, a polypeptide or polypeptide derivative can be formulated into or with liposomes, such as neutral or anionic liposomes, microspheres, ISCOMS, or virus-like particles (VLPs), to facilitate delivery and/or enhance the immune response. These compounds are readily available to those skilled in the art; for example, see Liposomes: A Practical Approach (supra). Adjuvants other than liposomes can also be used in the invention and are well known in the art (see, for example, the list provided below).

Administration can be achieved in a single dose or repeated as necessary at intervals that can be determined by one skilled in the art. For example, a priming dose can be followed by three booster doses at weekly or monthly intervals. An appropriate dose depends on various parameters, including the nature of the recipient (e.g., whether the recipient is an adult or an infant), the particular vaccine antigen, the route and frequency of administration, the presence/absence or type of adjuvant, and the desired effect (e.g., protection and/or treatment), and can be readily determined by one skilled in the art. In general, a vaccine antigen of the invention can be administered mucosally in an amount ranging from about 10 μg to about 500 mg, preferably from about 1 mg to about 200 mg. For a parenteral route of administration, the dose usually should not exceed about 1 mg, and is, preferably, about 100 μg.

When used as components of a vaccine, the polynucleotides and polypeptides of the invention can be used sequentially as part of a multi-step immunization process. For example, a mammal can be initially primed with a vaccine vector of the invention, such as a pox virus, e.g., via a parenteral route, and then boosted twice with a polypeptide encoded by the vaccine vector, e.g., via the mucosal route. In another example, liposomes associated with a polypeptide or polypeptide derivative of the invention can be used for priming, with boosting being carried out mucosally using a soluble polypeptide or polypeptide derivative of the invention, in combination with a mucosal adjuvant (e.g., LT).

Polypeptides and polypeptide derivatives of the invention can also be used as diagnostic reagents for detecting the presence of anti-Helicobacter antibodies, e.g., in blood samples. Such polypeptides can be about 5 to about 80, preferably, about 10 to about 50 amino acids in length and can be labeled or unlabeled, depending upon the diagnostic method. Diagnostic methods involving such a reagent are described below.

Upon expression of a polynucleotide molecule of the invention, a polypeptide or polypeptide derivative is produced and can be purified using known methods. For example, the polypeptide or polypeptide derivative can be produced as a fusion protein containing a fused tail that facilitates purification. The fusion product can be used to immunize a small mammal, e.g., a mouse or a rabbit, in order to raise monospecific antibodies against the polypeptide or polypeptide derivative. The eighth aspect of the invention thus provides a monospecific antibody that binds to a polypeptide or polypeptide derivative of the invention.

By “monospecific antibody” is meant an antibody that is capable of reacting with a unique, naturally-occurring Helicobacter polypeptide. An antibody of the invention can be polyclonal or monoclonal. Monospecific antibodies can be recombinant, e.g., chimeric (e.g., consisting of a variable region of murine origin and a human constant region), humanized (e.g., a human immunoglobulin constant region and a variable region of animal, e.g., murine, origin), and/or single chain. Both polyclonal and monospecific antibodies can also be in the form of immunoglobulin fragments, e.g., F(ab)′2 or Fab fragments. The antibodies of the invention can be of any isotype, e.g., IgG or IgA, and polyclonal antibodies can be of a single isotype or can contain a mixture of isotypes.

The antibodies of the invention, which can be raised to a polypeptide or polypeptide derivative of the invention, can be produced and identified using standard immunological assays, e.g., Western blot assays, dot blot assays, or ELISA (see, e.g., Coligan et al., Current Protocols in Immunology, John Wiley & Sons, Inc., New York, N.Y., 1994). The antibodies can be used in diagnostic methods to detect the presence of Helicobacter antigens in a sample, such as a biological sample. The antibodies can also be used in affinity chromatography methods for purifying a polypeptide or polypeptide derivative of the invention. As is discussed further below, the antibodies can also be used in prophylactic and therapeutic passive immunization methods.

Accordingly, a ninth aspect of the invention provides (i) a reagent for detecting the presence of Helicobacter in a biological sample that contains an antibody, polypeptide, or polypeptide derivative of the invention; and (ii) a diagnostic method for detecting the presence of Helicobacter in a biological sample, by contacting the biological sample with an antibody, a polypeptide, or a polypeptide derivative of the invention, so that an immune complex is formed, and detecting the complex as an indication of the presence of Helicobacter in the sample or the organism from which the sample was derived. The immune complex is formed between a component of the sample and the antibody, polypeptide, or polypeptide derivative, and that any unbound material can be removed prior to detecting the complex. A polypeptide reagent can be used for detecting the presence of anti-Helicobacter antibodies in a sample, e.g., a blood sample, while an antibody of the invention can be used for screening a sample, such as a gastric extract or biopsy sample, for the presence of Helicobacter polypeptides.

For use in diagnostic methods, the reagent (e.g., the antibody, polypeptide, or polypeptide derivative of the invention) can be in a free state or can be immobilized on a solid support, such as, for example, on the interior surface of a tube or on the surface, or within pores, of a bead. Immobilization can be achieved using direct or indirect means. Direct means include passive adsorption (i.e., non-covalent binding) or covalent binding between the support and the reagent. By “indirect means” is meant that an anti-reagent compound that interacts with the reagent is first attached to the solid support. For example, if a polypeptide reagent is used, an antibody that binds to it can serve as an anti-reagent, provided that it binds to an epitope that is not involved in recognition of antibodies in biological samples. Indirect means can also employ a ligand-receptor system, for example, a molecule, such as a vitamin, can be grafted onto the polypeptide reagent and the corresponding receptor can be immobilized on the solid phase. This concept is illustrated by the well known biotin-streptavidin system. Alternatively, indirect means can be used, e.g., by adding to the reagent a peptide tail, chemically or by genetic engineering, and immobilizing the grafted or fused product by passive adsorption or covalent linkage of the peptide tail.

According to a tenth aspect of the invention, there is provided a process for purifying, from a biological sample, a polypeptide or polypeptide derivative of the invention, which involves carrying out antibody-based affinity chromatography with the biological sample, wherein the antibody is a monospecific antibody of the invention.

For use in a purification process of the invention, the antibody can be polyclonal or monospecific, and preferably is of the IgG type. Purified IgGs can be prepared from an antiserum using standard methods (see, e.g., Coligan et al., supra). Conventional chromatography supports, as well as standard methods for grafting antibodies, are described, for example, by Harlow et al. (Antibodies: A Laboratory Manual, Cold Spring Harbor Laboratory Press, Cold Spring Harbor, N.Y., 1988).

Briefly, a biological sample, such as an H. pylori extract, preferably in a buffer solution, is applied to a chromatography material, which is, preferably, equilibrated with the buffer used to dilute the biological sample, so that the polypeptide or polypeptide derivative of the invention (i.e., the antigen) is allowed to adsorb onto the material. The chromatography material, such as a gel or a resin coupled to an antibody of the invention, can be in batch form or in a column. The unbound components are washed off and the antigen is eluted with an appropriate elution buffer, such as a glycine buffer, a buffer containing a chaotropic agent, e.g., guanidine HCl, or a buffer having high salt concentration (e.g., 3 M MgCl₂). Eluted fractions are recovered and the presence of the antigen is detected, e.g., by measuring the absorbance at 280 nm.

An antibody of the invention can be screened for therapeutic efficacy as follows. According to an eleventh aspect of the invention, there is provided (i) a composition of matter containing a monospecific antibody of the invention, together with a diluent or carrier; (ii) a pharmaceutical composition containing a therapeutically or prophylactically effective amount of a monospecific antibody of the invention, and (iii) a method for treating or preventing Helicobacter (e.g., H. pylori, H. felis, H. mustelae, or H. heilmanii) infection, by administering a therapeutic or prophylactic amount of a monospecific antibody of the invention to an individual in need of such treatment. In addition, the eleventh aspect of the invention includes the use of a monospecific antibody of the invention in the preparation of a medicament for treating or preventing Helicobacter infection.

The monospecific antibody can be polyclonal or monoclonal, and is, preferably, predominantly of the IgA isotype. In passive immunization methods, the antibody is administered to a mucosal surface of a mammal, e.g., the gastric mucosa, e.g., orally or intragastrically, optionally, in the presence of a bicarbonate buffer. Alternatively, systemic administration, not requiring a bicarbonate buffer, can be carried out. A monospecific antibody of the invention can be administered as a single active agent or as a mixture with at least one additional monospecific antibody specific for a different Helicobacter polypeptide. The amount of antibody and the particular regimen used can be readily determined by one skilled in the art. For example, daily administration of about 100 to 1,000 mg of antibody over one week, or three doses per day of about 100 to 1,000 mg of antibody over two or three days, can be effective regimens for most purposes.

Therapeutic or prophylactic efficacy can be evaluated using standard methods in the art, e.g., by measuring induction of a mucosal immune response or induction of protective and/or therapeutic immunity, using, e.g., the H. felis mouse model and the procedures described by Lee et al. (Eur. J. Gastroenterology & Hepatology 7:303, 1995) or Lee et al. (J. Infect. Dis. 172:161, 1995). Those skilled in the art will recognize that the H. felis strain of the model can be replaced with another Helicobacter strain. For example, the efficacy of polynucleotide molecules and polypeptides from H. pylori is, preferably, evaluated in a mouse model using an H. pylori strain. Protection can be determined by comparing the degree of Helicobacter infection in the gastric tissue assessed by, for example, urease activity, bacterial counts, or gastritis, to that of a control group. Protection is shown when infection is reduced by comparison to the control group. Such an evaluation can be made for polynucleotides, vaccine vectors, polypeptides, and polypeptide derivatives, as well as for antibodies of the invention.

For example, various doses of an antibody of the invention can be administered to the gastric mucosa of mice previously challenged with an H. pylori strain, as described, e.g., by Lee et al. (supra). Then, after an appropriate period of time, the bacterial load of the mucosa can be estimated by assessing urease activity, as compared to a control. Reduced urease activity indicates that the antibody is therapeutically effective.

Adjuvants that can be used in any of the vaccine compositions described above are described as follows. Adjuvants for parenteral administration include, for example, aluminum compounds, such as aluminum hydroxide, aluminum phosphate, and aluminum hydroxy phosphate. The antigen can be precipitated with, or adsorbed onto, the aluminum compound using standard methods. Other adjuvants, such as RIBI (ImmunoChem, Hamilton, Mont.), can also be used in parenteral administration.

Adjuvants that can be used for mucosal administration include, for example, bacterial toxins, e.g., the cholera toxin (CT), the E. coli heat-labile toxin (LT), the Clostridium difficile toxin A, the pertussis toxin (PT), and combinations, subunits, toxoids, or mutants thereof. For example, a purified preparation of native cholera toxin subunit B (CTB) can be used. Fragments, homologs, derivatives, and fusions to any of these toxins can also be used, provided that they retain adjuvant activity. Preferably, a mutant having reduced toxicity is used. Suitable mutants are described, e.g., in WO 95/17211 (Arg-7-Lys CT mutant), WO 96/6627 (Arg-192-Gly LT mutant), and WO 95/34323 (Arg-9-Lys and Glu-129-Gly PT mutant). Additional LT mutants that can be used in the methods and compositions of the invention include, e.g., Ser-63-Lys, Ala-69-Gly, Glu-110-Asp, and Glu-112-Asp mutants. Other adjuvants, such as the bacterial monophosphoryl lipid A (MPLA) of, e.g., E. coli, Salmonella Minnesota, Salmonella typhimurium, or Shigella flexneri; saponins, and polylactide glycolide (PLGA) microspheres, can also be used in mucosal administration. Adjuvants useful for both mucosal and parenteral administrations, such as polyphosphazene (WO 95/2415), can also be used.

Any pharmaceutical composition of the invention, containing a polynucleotide, polypeptide, polypeptide derivative, or antibody of the invention, can be manufactured using standard methods. It can be formulated with a pharmaceutically acceptable diluent or carrier, e.g., water or a saline solution, such as phosphate buffer saline, optionally, including a bicarbonate salt, such as sodium bicarbonate, e.g., 0.1 to 0.5 M. Bicarbonate can advantageously be added to compositions intended for oral or intragastric administration. In general, a diluent or carrier can be selected on the basis of the mode and route of administration, and standard pharmaceutical practice. Suitable pharmaceutical carriers and diluents, as well as pharmaceutical necessities for their use in pharmaceutical formulations, are described in Remington's Pharmaceutical Sciences, a standard reference text in this field and in the USP/NF.

The invention also includes methods in which gastroduodenal infections, such as Helicobacter infection, are treated by oral administration of a Helicobacter polypeptide of the invention and a mucosal adjuvant, in combination with an antibiotic, an antisecretory agent, a bismuth salt, an antacid, sucralfate, or a combination thereof. Examples of such compounds that can be administered with the vaccine antigen and an adjuvant are antibiotics, including, e.g., macrolides, tetracyclines, β-lactams, aminoglycosides, quinolones, penicillins, and derivatives thereof (specific examples of antibiotics that can be used in the invention include, e.g., amoxicillin, clarithromycin, tetracycline, metronidizole, erythromycin, cefuroxime, and erythromycin); antisecretory agents, including, e.g., H₂-receptor antagonists (e.g., cimetidine, ranitidine, famotidine, nizatidine, and roxatidine), proton pump inhibitors (e.g., omeprazole, lansoprazole, and pantoprazole), prostaglandin analogs (e.g., misoprostil and enprostil), and anticholinergic agents (e.g., pirenzepine, telenzepine, carbenoxolone, and proglumide); and bismuth salts, including colloidal bismuth subcitrate, tripotassium dicitrate bismuthate, bismuth subsalicylate, bicitropeptide, and pepto-bismol (see, e.g., Goodwin et al., Helicobacter pylori, Biology and Clinical Practice, CRC Press, Boca Raton, Fla., pp 366-395, 1993; Physicians' Desk Reference, 49^{th} edn., Medical Economics Data Production Company, Montvale, N.J., 1995). In addition, compounds containing more than one of the above-listed components coupled together, e.g., ranitidine coupled to bismuth subcitrate, can be used. The invention also includes compositions for carrying out these methods, i.e., compositions containing a Helicobacter antigen (or antigens) of the invention, an adjuvant, and one or more of the above-listed compounds, in a pharmaceutically acceptable carrier or diluent.

Amounts of the above-listed compounds used in the methods and compositions of the invention can readily be determined by one skilled in the art. In addition, one skilled in the art can readily design treatment/immunization schedules. For example, the non-vaccine components can be administered on days 1-14, and the vaccine antigen+adjuvant can be administered on days 7, 14, 21, and 28.

Methods and pharmaceutical compositions of the invention can be used to treat or to prevent Helicobacter infections and, accordingly, gastroduodenal diseases associated with these infections, including acute, chronic, and atrophic gastritis, and peptic ulcer diseases, e.g., gastric and duodenal ulcers.

A 76 kDa protein band containing GHPO 386, GHPO 789, and GHPO 1516 (hereinafter the “purified 76 kDa proteins”) was purified from Helicobacter pylori strain ATCC number 43579 (American Type Culture Collection, Rockville, Md.) by immunoaffinity-based chromatography using the methods described below in Example 1, and the purified 76 kDa proteins were shown to be effective vaccine antigens as follows.

Groups of 10 mice each were orally immunized with 1, 5, or 25 μg of the purified 76 kDa proteins, in combination with 5 μg of the heat-labile enterotoxin (LT) of E. coli. Twenty five μg of recombinant urease, in combination with 5 μg LT, was used as a positive control, and 5 μg of LT in PBS was used as a negative control. The immunizations were carried out four times each, on days 0, 7, 14, and 21 of the experiment. On day 33, blood samples were collected from the mice and, on day 34, saliva samples were collected. On day 35, all of the mice were challenged by intragastric administration of 1×10⁷ streptomycin-resistant, mouse-adapted H. pylori. On day 49, additional saliva samples were collected and, about two weeks after challenge, on days 52-53, the mice were sacrificed. Stomachs were removed from the mice and were analyzed for Helicobacter infection by measuring urease activity in the intact stomach tissue and by a quantitative culture study (Table 1).

Briefly, these studies showed that the gastric urease activities in samples from mice immunized with all three amounts of the purified 76 kDa proteins (i.e., 1, 5, and 25 μg), in combination with LT, were generally lower than the gastric urease activities of samples from mice immunized with LT alone or mice that were not treated prior to challenge. Levels of gastric urease activity generally decreased with increasing amounts of the purified 76 kDa proteins administered, with the gastric urease activity levels for the 25 μg doses of the purified 76 kDa proteins generally approaching those of mice immunized with 25 μg of recombinant urease and LT.

The quantitative culture analyses showed that the levels of Helicobacter detected in the stomachs of mice immunized with the purified 76 kDa proteins, which generally decreased with increasing dosages, were less than the levels detected in the stomachs of control mice that were immunized with LT alone or untreated before Helicobacter challenge (Table 1). The percentages of mice protected by immunization with the purified 76 kDa proteins approached the percentages of mice protected by treatment with urease (Table 1). These results show that the purified 76 kDa proteins are effective vaccine antigens for use in preventing Helicobacter infection.

TABLE 2
Prophylactic immunization with PMsv antigens as an oral dose response against an H. pylori challenge

| | BALB/c mice | Fisher's exact test | | |
|---|---|---|---|---|
| | # mice infected | infection status | | |
| | ANTRUM | based on quant. A550 ratios | | Wilcoxon rank sums test |
| | based on quant. A550 | Tx. gr. vs. LT only (gr.11) | CFU/ml (¼ antrum) | CFU Tx. gr. vs. LT control (gr.11) |
| Tx | 0.148 O.D. cutoff | p -value | MEAN ± SD | p -value |
| 1 μg 76 kDa + LT | 56% | 0.1409 | 39922 ± 34708 | 0.2203 |
| | (5/9) | | | |
| 5 μg 76 kDa + LT | 80% | 1 | 8802 ± 7788 | 0.0864 |
| | (4/5) | | | |
| 25 μg 76 kDa + LT | 33% | 0.0198 | 9712 ± 12183 | 0.0178 |
| | (3/9) | | | |
| 25 μg rUre + LT | 0 | 0.0001 | 8208 ± 8021 | 0.0179 |
| | (0/9) | | | |
| LT | 90% | | 107340 ± 127949 | |
| | (9/10) | | | |
| | 90% | not determined | 46173 ± 42325 | 0.2568 |
| | (9/10) | | | |

The invention is further illustrated by the following examples. Example 1 describes purification GHPO 1516, which is a 76 kDa protein, from Helicobacter cultures. Example 2 describes identification of genes, e.g., genes encoding 76 kDa proteins such as GHPO 386, GHPO 789, GHPO 1516, GHPO 1197, GHPO 1180, GHPO 896, GHPO 711, GHPO 190, GHPO 185, GHPO 1417, and GHPO 1414, in the Helicobacter genome, as well as identification of leader sequences, and primer design for amplification of genes lacking signal sequences. Example 3 describes cloning of DNA encoding GHPO 386, GHPO 789, GHPO 1516, and GHPO 896 into a vector that provides a histidine tag, and production and 10 purification of the resulting his-tagged fusion proteins. Example 4 describes methods for cloning DNA encoding the polypeptides of the invention so that they can be produced without His-tags, and Example 5 describes methods for purifying recombinant polypeptides of the invention.

### EXAMPLE 1

Purification and Partial Sequence Analysis of GHPO 1516, a 76 kDa Protein, from Helicobacter pylori

1.A. Culture and Initial Purification Steps

Frozen seeds from H. pylori strain ATCC 43579 are used to seed a 75 cm² flask containing a biphasic medium (a solid phase made of Colombia gelose containing 6% fresh sheep blood and a liquid phase made of triptcase soja containing 20% fetal calf serum). After 24 hours of culturing under microaerophilic conditions, the liquid phase is used for seeding several 75 cm² flasks containing biphasic medium lacking sheep blood. After 24 hours of culture, the liquid phase is used to seed a 2 L biofermentor in triptcase soja liquid phase containing 10 g/L beta-cyclodextrine. At OD 1.5-1.8, this culture is diluted in a 10 L biofermentor containing the liquid medium. After 24 hours, the bacteria are centrifuged at 4,000× g for 30 minutes at 4° C. A 10 L culture contains about 20 to 30 g (wet weight) bacteria.

The pellet obtained using the method described above is washed with 500 mL Phosphate Buffer Saline (PBS: 7.650 g NaCl, 0.724 g disodium phosphate, and 0.210 g monopotassium phosphate for one liter (pH 7.2)) for a one liter culture. The bacteria are then centrifuged again under the same conditions.

The pellet (C1) is suspended in 1% N-octyl-D-glucopyranoside (NOG; 30 mL/L; Sigma). The bacterial suspension is incubated for 1 hour at room temperature under stirring, centrifuged at 17,600× g for 30 minutes at 4° C., and the pellet (C2) is recovered.

The supernatant (S2) is dialyzed against PBS overnight at 4° C. under stirring. The precipitate is recovered by centrifugation at 2,600× g for 30 minutes at 4° C. The supernatant (S2d) is discarded and the pellet (Cs2d) is recovered and stored at −20° C.

The pellet (C2) is resuspended in 20 mM Tris-HCl buffer (pH 7.5) and 100 FM Pefabloc (Buffer A), and is homogenized with an ultra-turrax (3821, Janke and Kungel). Lysozyme and EDTA are added at 0.1 mg/mL and 1 mM, respectively.

The homogenate is sonicated three times for 2 minutes each at 4° C., and then is spun in an ultracentrifuge at 210,000× g for 30 minutes at 4° C. The supernatant (S3), which contains the cytoplasmic and periplasmic proteins, is eliminated, while the pellet is recovered, washed with buffer A, and spun in an ultracentrifuge at 210,000× g for 30 minutes at 4° C. The supernatant (S4) is eliminated and the pellet (C4) is stored at −20° C. This pellet (C4) contains membrane proteins.

The pellet is washed in 50 mM NaCO₃ (pH 9.5) and 100 μM Pefabloc (buffer B). The suspension is spun in an ultracentrifuge at 210,000× g for 30 minutes at 4° C. The supernatant (S5) is eliminated, and the pellet (C5) is then washed and spun in an ultracentrifuge as is described above. The supernatant (S6) is eliminated and the pellet (C6) is stored at −20° C.

1.B. Purification of the Proteins of Membrane Fraction C4 by Preparative SDS-PAGE

SDS-PAGE is carried out according to the method of Laemmli (supra), using a biphasic gel consisting of a 5% polyacrylamide concentrating gel and a 10% polyacrylamide separating gel. The membrane fraction C4 is resuspended in buffer A, diluted in an equal volume of 2× sample buffer, and heated for 5 minutes at 95° C. About 19 mg of protein is applied to the gel (16×12 cm; thickness: 5 mm). Pre-migration is carried out for 2 hours at 50 V, and is followed by migration overnight at 65 V. After Coomassie blue staining, five major bands are revealed that have apparent molecular weights of 87, 76, 54, 50, and 32 kD. Bands at 50 and 32 kDa appear to be slightly contaminated with bands at 47 and 35 kD, respectively.

A band corresponding to the purified 76 kDa proteins is cut out from the gel and is pounded with an ultra-turrax in 10-20 ML extraction buffer (25 mM Tris-HCl (pH 8.8), 8 M urea, 10% SDS, 100 μM phenyl methyl sulfonyl fluoride (PMSF), and 10 μM Pefabloc (buffer C)).

Each homogenate is filtered through a Millipore AP20 filter under 7 bars at room temperature, washed with 5-10 mL buffer C, and then filtered again. Each filtrate is precipitated with three volumes of a 50/50 mixture of 75% methanol and 75% isopropanol, and then is centrifuged at 240,000× g for 16 hours at 10° C.

Each pellet is resuspended in 2 mL of 10 mM NaPO₄ (pH 7.0) containing 1 M NaCl, 0.1% Sarkosyl, 100 ptM PMSF, and 6 M urea (buffer D).

The solubilized sample is dialyzed, in order, against 100 mL buffer D containing 4 M urea, 100 mL buffer D containing 2 M urea and 0.5% Sarkosyl, and twice against 100 mL buffer D that does not contain urea or Sarkosyl. The dialyses are carried out for 1 hour each under stirring at room temperature. The last dialysate is incubated for 30 minutes in an ice bath, and then is centrifuged at low speed for 10 minutes at 4° C. The supernatant is recovered, filtered through a Millipore filter (0.45 em), and stored at −20° C.

1.C. Purification of the 76 kDa Protein by Immunoaffinity-based Chromatography

1.C.1. Antiserum Preparation

Specific polyclonal serum against the purified 76 kDa proteins, which are purified by preparative SDS-PAGE, is prepared by hyperimmunizing rabbits as follows. On day 0, a preparation containing 50 fig of the protein mixed with complete Freund's adjuvant is administered subcutaneously to the rabbits at multiple sites. The rabbits are boosted at days 21 and 42 with 25 μg of the protein in incomplete Freund's adjuvant, and are sacrificed at day 60. Complement is removed from the serum by heating for 30 minutes at 56° C. The hyperimmune serum is then sterilized by filtration through a Millipore membrane (0.22 μm).

1.C.2. IgG Purification

The hyperimmune serum prepared as described above is applied to a Protein A Sepharose Fast Flow column (Pharmacia) that is equilibrated with 100 mM Tris-HCl (pH 8.0). The column is washed with 10 column volumes of 100 mM Tris-HCl (pH 8.0), and then with 10 column volumes of 10 mM Tris-HCl (pH 8.0). IgGs are eluted in 0.1 M glycine buffer (pH 3.0), and are collected as 5 mL fractions, to each of which 0.25 mL of Tris-HCl (pH 8.0) is added. The optical density is measured at 280 nm, the IgG-containing fractions are pooled together, and, if necessary, frozen at −70° C.

1.C.3. Preparation of the Column

An appropriate amount of CNBr-activated Sepharose 4B gel (Pharmacia; reference: 17-0430-01) is suspended in 1 mM NaCl buffer (1 g dry gel provides for 3.5 mL hydrated gel; 5 to 10 mg IgGs can be retained per mL of hydrated gel). The gel is then washed with a buchner by adding small quantities of 1 mM HCl. The total volume of 1 mM HCl that is used amounts to 200 mL/g of gel.

Purified IgGs are dialyzed for 4 hours at room temperature against 50 volumes of 500 mM sodium phosphate buffer (pH 7.5). The IgGs are then diluted to 3 mg/mL with the same buffer. IgGs are incubated with the gel overnight at 5±3° C. under stirring. The gel is packed in a chromatography column and is washed with 2 column volumes of 500 mM phosphate buffer (pH 7.5). The gel is then transferred to a tube and is incubated with 100 mM ethanolamine (pH 7.5), and then it is washed with 2 column volumes of PBS. The gel can be stored in PBS/merthiolate, 1/10,000.

1.C.4. Adsorption and Elution

The membrane fraction Cs2d is suspended in 50 mM Tris-HCl (pH 8.0), 2 mM EDTA, and then is filtered through a 0.45 μm membrane. The supernatant is applied to the column, which is equilibrated with 50 mM Tris-HCl (pH 8.0), 2 mM EDTA, at a flow rate of about 10 mL/hour. The column is washed with 20 column volumes of 50 mM Tris-HCl (pH 8.0), 2 mM EDTA, and then with 2 to 6 volumes 10 mM phosphate buffer (pH 6.8).

The antigen is eluted with 100 mM glycine buffer (pH 2.5). The eluate is collected in 3 mL fractions, to each of which is added 150 μL 1 M phosphate buffer (pH 8.0). The optical density of each fraction is measured at 280 nm, fractions containing the 76 kDa protein are pooled, and stored at −70° C.

Analysis by 10% SDS-PAGE reveals a single band at 76 kD. N-terminal sequence was carried out on this purified 76 kDa preparation, and the sequence obtained is as follows: EDDGFYTSVGYQIGEAAQMV (SEQ ID NO:58).

1.D. Purification of the 76 kDa Protein from Membrane Fraction Cs2d

The 76 kDa protein can also be purified as follows. A 40 mL Q-Sepharose column (diameter: 2.5 cm; height: 8 cm) is prepared according to the manufacturer's instructions (Pharmacia). The column is washed and equilibrated with buffer B, containing 50 mM NaCO₃ (pH 9.5), 100 μM Pefabloc, and 0.1% Zwiftergent 3-14. The chromatography is monitored by measuring absorbance at 280 nm at the column exit.

One hundred and forty mg of protein from the membrane fraction Cs2d resuspended in buffer B are applied to the column. The column is washed with 0.1 M NaCl in buffer B, and then a 0.1-0.5 M NaCl gradient is applied to the column. The fraction eluted between 0.35 and 0.45 M NaCl is further purified on a 10 mL S-Sepharose column (diameter: 1.5 cm; height: 5 cm; up to 10 mg protein/mL of gel), which is prepared according to the manufacturer's instructions (Pharmacia). The fraction obtained is dialyzed against 50 mM acetate (pH 5.0) containing 100 μM Pefabloc and 0.1% Zwittergent 3-14, and then is applied to the column, which is equilibrated with the acetate buffer.

The column is washed with the acetate buffer until the absorbance at 280 nm is stabilized (about 3 column volumes are required). Proteins are eluted with a 0-0.5 M NaCl gradient in acetate buffer. The fraction eluted at 0.15 M NaCl is enriched with the 76 kDa protein.

### EXAMPLE 2

Identification of Genes in the H. pylori Genome, Such as Genes Encoding the 76 kDa Proteins, Identification of Leader Sequences, and Primer Design for Amplification of Genes Lacking Signal Sequences

2.A. Creating H. pylori Genomic Databases

The H. pylori genome was provided as a text file containing a single contiguous string of nucleotides that had been determined to be 1.76 Megabases in length. The complete genome was split into 17 separate files using the program SPLIT (Creativity in Action), giving rise to 16 contigs, each containing 100,000 nucleotides, and a 17^{th} contig containing the remaining 76,000 nucleotides. A header was added to each of the 17 files using the format: >hpg0.txt (representing contig 1), .hpgl.txt (representing contig 2), etc. The resulting 17 files, named hpg0 through hpg16, were then copied together to form one file that represented the plus strand of the complete H. pylori genome. The constructed database was given the designation “H.” A negative strand database of the H. pylori genome was created similarly by first creating a reverse complement of the positive strand using the program SeqPup (D. G. Gilbert, Indiana University Biology Department) and then performing the same procedure as described above for the plus strand. This database was given the designation “N.”

The regions predicted to encode open reading frames (ORFs) were defined for the complete H. pylori genome using the program GENEMARK™ (Borodovsky et al., Comp. Chem. 17:123, 1993). A database was created from a text file containing an annotated version of all ORFs predicted to be encoded by the H. pylori genome for both the plus and minus strands, and was given the designation “O.” Each ORF was assigned a number indicating its location on the genome and its position relative to other genes. No manipulation of the text file was required.

2.B. Searching the H. pylori Databases

The databases constructed as is described above were searched using the program FASTA (Pearson et al., Proc. Natl. Acad. Sci. USA 85:2444-2448, 1988). FASTA was used for searching either a DNA sequence against either of the gene databases (“H” and/or “N”), or a peptide sequence against the ORF library (“O”). TFASTX was used to search a peptide sequence against all possible reading frames of a DNA database (“H” and/or “N” libraries). Potential frameshifts also being resolved, FASTX was used for searching the translated reading frames of a DNA sequence against either a DNA database, or a peptide sequence against the protein database.

2.C. Isolation of DNA Sequences from the H. pylori Genome

The FASTA searches against the constructed DNA databases identified exact nucleotide coordinates on one or more of the isolated contigs, and therefore the location of the target DNA. Once the exact location of the target sequence was known, the contig identified to carry the gene was exported into the software package MapDraw (DNAStar, Inc.) and the gene was isolated. Gene sequences with flanking DNA was then excised and copied into the EditSeq. Software package (DNAStar, Inc.) for further analysis.

2.D. Identification of Leader Sequences

The deduced protein encoded by a target gene sequence is analyzed using the PROTEAN software package (DNAStar, Inc.). This analysis predicts those areas of the protein that are hydrophobic by using the Kyte-Dooliffle algorithm, and identifies any potential polar residues preceding the hydrophobic core region, which is typical for many leader sequences. For confirmation, the target protein is then searched against a PROSITE database (DNAStar, Inc.) consisting of motifs and signatures. Characteristic of many leader sequences and hydrophobic regions in general, is the identification of predicted prokaryotic lipid attachment sites. Where confirmation between the two approaches is apparent at the N-terminus of any protein, putative cleavage sites are sought. Specifically, this includes the presence of either an Alanine (A), Serine (S), or Glycine (G) residue immediately after the core hydrophobic region. In the case of lipoproteins, a Cysteine (C) residue would be identified as the +1 residue, post-cleavage.

2.E. Rational Design of PCR Primers Based on the Identification of Leader Sequences

In order to clone gene sequences as N-terminus translational fusions for the generation of recombinant proteins with N-terminal Histidine tags, the gene sequence that specifies the leader sequence is omitted. The 5′-end of the gene-specific portion of the N-terminal primer is designed to start at the first codon beyond the cleavage site. In the case of lipoproteins, the 5′-end of the N-terminal primer begins at the second codon, immediately after the modifiable residue at position +1 post-cleavage. The omission of the leader sequence from the recombinant allows for one-step purification, and potential problems associated with insertion of leader sequences in the membrane of the host strain carrying the hybrid construct are avoided.

### EXAMPLE 3

Preparation of Isolated DNA Encoding GHPO 386, GHPO 789, GHPO 1516, and GHPO 896 and Production of these Proteins as a Histidine-tagged Fusion Proteins

3.A. Preparation of Genomic DNA from Helicobacter pylori

Helicobacter pylori strain ORV2001, stored in LB medium containing 50% glycerol at −70° C., is grown on Colombia agar containing 7% sheep blood for 48 hours under microaerophilic conditions (8-10% CO₂, 5-7% O₂, 85-87% N₂). Cells are harvested, washed with phosphate buffer saline (PBS) (pH 7.2), and DNA is then extracted from the cells using the Rapid Prep Genomic DNA Isolation kit (Pharmacia Biotech).

3.B. PCR Amplification

DNA encoding GHPO 386, GHPO 789, GHPO 1516, and GHPO 896 is amplified from genomic DNA, as can be prepared as is described above, by the Polymerase Chain Reaction (PCR) using the following primers:

| | |
|---|---|
| GHPO 386: | |
| N-terminal primer: | |
| 5′-CTGAATTCGATTTCAAGGAGAAAACATGAAA-3′ | (SEQ ID NO:59); and |
| C-terminal primer: | |
| 5′-CCGCTCGAGTTAGTAAGCGAACACATAATT-3′ | (SEQ ID NO:60). |
| GHPO 789: | |
| N-terminal primer: | |
| 5′-CGCGGATCCGAATCCAATTTAATCCAAAAAGG-3′ | (SEQ ID NO:61); and |
| C-terminal primer: | |
| 5′-CCGCTCGAGTTAGTAAGCGAACACATAGTTCAA-3′ | (SEQ ID NO:62). |
| GHPO 1516: | |
| N-terminal primer: | |
| 5′-CGCGGATCCGAATCCAATTTAATCCAAAAAGG-3′ | (SEQ ID NO:56); and |
| C-terminal primer: | |
| 5′-CCGCTCGAGTTAAGTAAGCGAACACATATTCAA-3′ | (SEQ ID NO: 57). |
| GHPO 896: | |
| N-terminal primer: | |
| 5′-CGCGGATCCGAAGTTTCTTTGTATCAAAG-3′ | (SEQ ID NO:63); and |
| C-terminal primer: | |
| 5′-CCGCTCGAGTTAGTAAGCAAACACATAATTGTG-3′ | (SEQ ID NO:64). |

The N-terminal and C-terminal primers each include a 5′ clamp and a restriction enzyme recognition sequence for cloning purposes (BamHI (GGATCC) and XhoI (CTCGAG) recognition sequences). Amplification of gene-specific DNA is carried out using a heat-stable DNA Polymerase according to the manufacturer's instructions. The reaction mixture, which is brought to a final volume of 100 μL with distilled water, is as follows:

| | | |
|---|---|---|
| dNTPs mix | 200 | μM |
| 10x ThermoPol buffer | 10 | μL |
| primers | 300 | nM each |
| DNA template | 50 | ng |
| DNA polymerase | 2 | units |

Appropriate amplification reaction conditions can readily be determined by one skilled in the art. In the present case, the following conditions were used. For GHPO 386 and GHPO 789, in a reaction containing Taq DNA polymerase (Appligene), a denaturing step was carried out at 95° C. for 30 seconds, followed by an annealing step at 50° C. for one minute, and an extension step at 72° C. for 2 minutes and 30 seconds. Twenty five cycles were carried out. For GHPO 896, in a reaction containing Taq DNA polymerase, a denaturing step was carried out at 97° C. for 30 seconds, followed by an annealing step at 50° C. for one minute, and an extension step at 72° C. for 2 minutes and 30 seconds. Twenty five cycles were carried out. The same reaction conditions were used for GHPO 1516 as GHPO 896, except that Vent DNA polymerase was used for clone GHPO 1516, instead of Taq DNA polymerase, and the annealing temperature was 55° C.

3.C. Transformation and Selection of Transformants

A single PCR product is thus amplified and is then digested at 37° C. for 2 hours with BamHI and XhoI concurrently in a 20 μL reaction volume. The digested product is ligated to similarly cleaved pET28a (Novagen) that is dephosphorylated prior to the ligation by treatment with Calf Intestinal Alkaline Phosphatase (CIP). The gene fusion constructed in this manner allows one-step affinity purification of the resulting fusion protein because of the presence of histidine residues at the N-terminus of the fusion protein, which are encoded by the vector.

The ligation reaction (20 μL) is carried out at 14° C. overnight and then is used to transform 100 μL fresh E. coli XL 1-blue competent cells (Novagen). The cells are incubated on ice for 2 hours, then heat-shocked at 42° C. for 30 seconds, and returned to ice for 90 seconds. The samples are then added to 1 mL LB broth in the absence of selection and grown at 37° C. for 2 hours. The cells are then plated out on LB agar containing kanamycin (50 μg/mL) at a 10× and neat dilution and incubated overnight at 37° C. The following day, 50 colonies are picked onto secondary plates and incubated at 37° C. overnight.

Five colonies are picked into 3 mL LB broth supplemented with kanamycin (100 μg/mL) and are grown overnight at 37° C. Plasmid DNA is extracted using the Quiagen mini-prep. method and is quantitated by agarose gel electrophoresis.

PCR is performed with the gene-specific primers under the conditions stated above and transformant DNA is confirmed to contain the desired insert. If PCR-positive, one of the five plasmid DNA samples (500 ng) extracted from the E. coli XL1-blue cells is used to transform competent BL21 (ADE3) E. coli competent cells (Novagen; as described previously). Transformants (10) are picked onto selective kanamycin (50 μg/mL) containing LB agar plates and stored as a research stock in LB containing 50% glycerol.

3.D. Purification of Recombinant Proteins

One mL of frozen glycerol stock prepared as described in 3.C. is used to inoculate 50 mL of LB medium containing 25 μg/mL of kanamycin in a 250 mL Erlenmeyer flask. The flask is incubated at 37° C. for 2 hours or until the absorbance at 600 nm (OD₆₀₀) reaches 0.4-1.0. The culture is stopped from growing by placing the flask at 4° C. overnight. The following day, 10 mL of the overnight culture are used to inoculate 240 mL LB medium containing kanamycin (25 μg/mL), with the initial OD₆₀₀ about 0.02-0.04. Four flasks are inoculated for each ORF.

The cells are grown to an OD₆₀₀ of 1.0 (about 2 hours at 37° C.), an 1 mL sample is harvested by centrifugation, and the sample is analyzed by SDS-PAGE to detect any leaky expression. The remaining culture is induced with 1 mM IPTG and the induced cultures are grown for an additional 2 hours at 37° C.

The final OD₆₀₀ is taken and the cells are harvested by centrifugation at 5,000× g for 15 minutes at 4° C. The supernatant is discarded and the pellets are resuspended in 50 mM Tris-HCl (pH 8.0), 2 mM EDTA. Two hundred and fifty mL of buffer are used for a 1 L culture and the cells are recovered by centrifugation at 12,000× g for 20 minutes. The supernatant is discarded and the pellets are stored at −45° C.

3. E. Protein Purification

Pellets obtained from 3.D. are thawed and resuspended in 95 mL of 50 mM Tris-HCl (pH 8.0). Pefabloc and lysozyme are added to final concentrations of 100 μM and 100 μg/mL, respectively. The mixture is homogenized with magnetic stirring at 5° C. for 30 minutes. Benzonase (Merck) is added at a 1 U/mL final concentration, in the presence of 10 mM MgCl₂, to ensure total digestion of the DNA. The suspension is sonicated (Branson Sonifier 450) for 3 cycles of 2 minutes each at maximum output. The homogenate is centrifuged at 19,000× g for 15 minutes and both the supernatant and the pellet are analyzed by SDS-PAGE to detect the cellular location of the target protein in the soluble or insoluble fractions, as is described further below.

3.E.1. Soluble Fraction

If the target protein is produced in a soluble form (i.e., in the supernatant obtained in 3.E.) NaCl and imidazole are added to the supernatant to final concentrations of 50 mM Tris-HCl (pH 8.0), 0.5 M NaCl, and 10 mM imidazole (buffer A). The mixture is filtered through a 0.45 μm membrane and loaded onto an IMAC column (Pharmacia HiTrap chelating Sepharose; 1 mL) that has been charged with nickel ions according to the manufacturer's recommendations. After loading, the column is washed with 50 column volumes of buffer A and the recombinant target protein is eluted with 5 mL of buffer B (50 mM Tris-HCl (pH 8.0), 0.5 M NaCl, 500 mM imidazole).

The elution profile is monitored by measuring the absorbance of the fractions at 280 nm. Fractions corresponding to the protein peak are pooled, dialyzed against PBS containing 0.5 M arginine, filtered through a 0.22 μm membrane, and stored at −45° C.

3.E.2. Insoluble Fraction

If the target protein is expressed in the insoluble fraction (pellets obtained from 3.E.), purification is conducted under denaturing conditions. NaCl, imidazole, and urea are added to the resuspended pellet to final concentrations of 50 mM Tris-HCl (pH 8.0), 0.5 M NaCl, 10 mM imidazole, and 6 M urea (buffer C). After complete solubilization, the mixture is filtered through a 0.45 μm membrane and loaded onto an IMAC column.

The purification procedures on the IMAC column are the same as described in 3.E.1., except that 6 M urea is included in all buffers used and 10 column volumes of buffer C are used to wash the column after protein loading, instead of 50 column volumes.

The protein fractions eluted from the IMAC column with buffer D (buffer C containing 500 mM imidazole) are pooled. Arginine is added to the solution to final concentration of 0.5 M and the mixture is dialyzed against PBS containing 0.5 M arginine and various concentrations of urea (4 M, 3 M, 2 M, 1 M, and 0.5 M) to progressively decrease the concentration of urea. The final dialysate is filtered through a 0.22 μm membrane and stored at −45° C.

Alternatively, when the above purification process is not as efficient as it should be, two other processes may be used as follows. A first alternative involves the use of a mild denaturant, N-octyl glucoside (NOG). Briefly, a pellet obtained in 3.E. is homogenized in 5 mM imidazole, 500 mM sodium chloride, 20 mM Tris-HCl (pH 7.9) by microfluidization at a pressure of 15,000 psi and is clarified by centrifugation at 4,000-5,000× g. The pellet is recovered, resuspended in 50 mM NaPO₄ (pH 7.5) containing 1-2% weight/volume NOG, and homogenized. The NOG-soluble impurities are removed by centrifugation. The pellet is extracted once more by repeating the preceding extraction step. The pellet is dissolved in 8 M urea, 50 mM Tris (pH 8.0). The urea-solubilized protein is diluted with an equal volume of 2 M arginine, 50 mM Tris (pH 8.0), and is dialyzed against 1 M arginine for 24-48 hours to remove the urea. The final dialysate is filtered through a 0.22 μm membrane and stored at −45° C.

A second alternative involves the use of a strong denaturant, such as guanidine hydrochloride. Briefly, a pellet obtained in 3.E. is homogenized in 5 mM imidazole, 500 mM sodium chloride, 20 mM Tris-HCl (pH 7.9) by microfluidization at a pressure of 15,000 psi and clarified by centrifugation at 4,000-5,000× g. The pellet is recovered, resuspended in 6 M guanidine hydrochloride, and passed through an IMAC column charged with Ni++. The bound antigen is eluted with 8 M urea (pH 8.5). Beta-mercaptoethanol is added to the eluted protein to a final concentration of 1 mM, then the eluted protein is passed through a Sephadex G-25 column equilibrated in 0.1 M acetic acid. Protein eluted from the column is slowly added to 4 volumes of 50 mM phosphate buffer (pH 7.0). The protein remains in solution.

3.F. Evaluation of the Protective Activity of the Purified Protein

A protection test is described above that was carried out for testing the protective activity of the purified, native proteins. This test can also be used for testing the protective efficacy of recombinant proteins. Alternatively, the following test can be used.

Groups of 10 OF1 mice (IFFA Credo) are immunized rectally with 25 μg of the purified recombinant protein, admixed with 1 μg of cholera toxin (Bema) in physiological buffer. Mice are immunized on days 0, 7, 14, and 21. Fourteen days after the last immunization, the mice are challenged with H. pylori strain ORV2001 grown in liquid media (the cells are grown on agar plates, as described in 1.A., and, after harvest, the cells are resuspended in Brucella broth; the flasks are then incubated overnight at 37° C.). Fourteen days after challenge, the mice are sacrificed and their stomachs are removed. The amount of H. pylori is determined by measuring the urease activity in the stomach and by culture.

3.G. Production of Monospecific Polyclonal Antibodies

3.G.1. Hyperimmune Rabbit Antiserum

New Zealand rabbits are injected both subcutaneously and intramuscularly with 100 μg of a purified fusion polypeptide, as obtained in 3.E.1. or 3.E.2., in the presence of Freund's complete adjuvant and in a total volume of approximately 2 mL. Twenty one and 42 days after the initial injection, booster doses, which are identical to priming doses, except that Freund's incomplete adjuvant is used, are administered in the same way. Fifteen days after the last injection, animal serum is recovered, decomplemented, and filtered through a 0.45 μm membrane.

3.G.2. Mouse Hyperimmune Ascites Fluid

Ten mice are injected subcutaneously with 10-50 μg of a purified fusion polypeptide as obtained in 3.E.1. or 3.E.2., in the presence of Freund's complete adjuvant and in a volume of approximately 200 μL. Seven and 14 days after the initial injection, booster doses, which are identical to the priming doses, except that Freund's incomplete adjuvant is used, are administered in the same way. Twenty one and 28 days after the initial infection, mice receive 50 μg of the antigen alone intraperitoneally. On day 21, mice are also injected intraperitoneally with sarcoma 180/TG cells CM26684 (Lennette et al., Diagnostic Procedures for Viral, Rickettsial, and Chlamydial Infections, 5th Ed. Washington D.C., American Public Health Association, 1979). Ascites fluid is collected 10-13 days after the last injection.

### EXAMPLE 4

Methods for Producing Transcriptional Fusions Lacking His-tags

Methods for amplification and cloning of DNA encoding the polypeptides of the invention as transcriptional fusions lacking His-tags are described as follows. Two PCR primers for each clone are designed based upon the sequences of the polynucleotides that encode them (SEQ ID NOs:1-21 (odd numbers)). These primers can be used to amplify DNA encoding the polypeptides of the invention from any Helicobacter pylori strain, including, for example, ORV2001 and the strain deposited with the American Type Culture Collection (ATCC, Rockville, Md.) as ATCC number 43579, as well as from other Helicobacter species.

The N-terminal primer is designed to include the ribosome binding site of the target gene, the ATG start site, the leader sequence, and the cleavage site. The N-terminal primers can include a 5′ clamp and restriction endonuclease recognition site, such as that for BamHI (GGATCC), which facilitates subsequent cloning. Similarly, the C-terminal primers can include a restriction endonuclease recognition site, such as that for XhoI (CTCGAG), which can be used in subsequent cloning, and a TAA stop codon. Specific primers that can be used are listed above.

Amplification of a genes encoding the polypeptides of the invention is carried out using Vent DNA polymerase (New England Biolabs) or Taq DNA polymerase (Appligene) under the conditions described above in Example 3. Alternatively, Thermalase DNA polymerase or Pwo DNA polymerase (Boehringer Mannheim) can be used, according to instructions provided by the manufacturers.

A single PCR product for each clone is amplified and can be cloned into BamHI-XhoI cleaved pET 24, resulting in construction of transcriptional fusions that permit expression of the proteins without His-tags. The expressed products can be purified as denatured proteins that are refolded by dialysis into 1 M arginine.

Cloning into pET 24 allows transcription of genes from the T7 promoter, which is supplied by the vector, but relies upon binding of the RNA-specific DNA polymerase to the intrinsic ribosome binding site of the genes, and thereby expression of the complete ORF. The amplification, digestion, and cloning protocols are as described above for constructing translational fusions.

### EXAMPLE 5

Purification of the Polypeptides of the Invention by Immunoaffinity

5.A. Purification of Specific IgGs

An immune serum, as prepared in section 3.G., is applied to a protein A Sepharose Fast Flow column (Pharmacia) equilibrated in 100 mM Tris-HCl (pH 8.0). The resin is washed by applying 10 column volumes of 100 mM Tris-HCl and 10 volumes of 10 mM Tris-HCl (pH 8.0) to the column. IgG antibodies are eluted with 0.1 M glycine buffer (pH 3.0) and are collected as 5 mL fractions to which is added 0.25 mL 1 M Tris-HCl (pH 8.0). The optical density of the eluate is measured at 280 nm and the fractions containing the IgG antibodies are pooled, dialyzed against 50 mM Tris-HCl (pH 8.0), and, if necessary, stored frozen at −70° C.

5.B. Preparation of the Column

An appropriate amount of CNBr-activated Sepharose 4B gel (1 g of dried gel provides for approximately 3.5 mL of hydrated gel; gel capacity is from 5 to 10 mg coupled IgG/mL of gel) manufactured by Pharmacia (17-0430-01) is suspended in 1 mM HCl buffer and washed with a buchner by adding small quantities of 1 mM HCl buffer. The total volume of buffer is 200 mL per gram of gel.

Purified IgG antibodies are dialyzed for 4 hours at 20+5° C. against 50 volumes of 500 mM sodium phosphate buffer (pH 7.5). The antibodies are then diluted in 500 mM phosphate buffer (pH 7.5) to a final concentration of 3 mg/mL.

IgG antibodies are mixed with the gel overnight at 5±3° C. The gel is packed into a chromatography column and is washed with 2 column volumes of 500 mM phosphate buffer (pH 7.5), and 1 column volume of 50 mM sodium phosphate buffer, containing 500 mM NaCl (pH 7.5). The gel is then transferred to a tube, mixed with 100 mM ethanolamine (pH 7.5) for 4 hours at room temperature, and washed twice with 2 column volumes of PBS. The gel is then stored in 1/10,000 PBS/merthiolate. The amount of IgG antibodies coupled to the gel is determined by measuring the optical density (OD) at 280 nm of the IgG solution and the direct eluate, plus washings.

5.C. Adsorption and Elution of the Antigen

An antigen solution in 50 mM Tris-HCl (pH 8.0), 2 mM EDTA, for example, the supernatant obtained in 3.E.1. or the solubilized pellet obtained in 3.E.2., after centrifugation and filtration through a 0.45 μm membrane, is applied to a column equilibrated with 50 mM Tris-HCl (pH 8.0), 2 mM EDTA, at a flow rate of about 10 mL/hour. The column is then washed with 20 volumes of 50 mM Tris-HCl (pH 8.0), 2 mM EDTA. Alternatively, adsorption can be achieved by mixing overnight at 5±3° C.

The adsorbed gel is washed with 2 to 6 volumes of 10 mM sodium phosphate buffer (pH 6.8) and the antigen is eluted with 100 mM glycine buffer (pH 2.5). The eluate is recovered in 3 mL fractions, to each of which is added 150 μL of 1 M sodium phosphate buffer (pH 8.0). Absorption is measured at 280 nm for each fraction; those fractions containing the antigen are pooled and stored at −20° C.

Other embodiments are within the following claims.

What is claimed is:

1. An isolated polynucleotide that encodes:
(i) a polypeptide comprising an amino acid sequence that is homologous to the amino acid sequence of a Helicobacter membrane-associated polypeptide, wherein said amino acid sequence of said Helicobacter membrane-associated polypeptide is selected from the group consisting of the amino acid sequences as shown:
in SEQ ID NO:2, beginning with an amino acid in any one of positions −19 to 5, preferably in position −19 or position 1, and ending with an amino acid in position 689 (GHPO 386);
in SEQ ID NO:4, beginning with an amino acid in any one of positions −20 to 5, preferably in position −20 or position 1, and ending with an amino acid in position 713 (GHPO 789);
in SEQ ID NO:6, beginning with an amino acid in any one of positions −20 to 5, preferably in position −20 or position 1, and ending with an amino acid in position 725 (GHPO 1516);
in SEQ ID NO:8, beginning with an amino acid in any one of positions −20 to 5, preferably in position −20 or position 1, and ending with an amino acid in position 691 (GHPO 1197);
in SEQ ID NO:10, beginning with an amino acid in any one of positions −20 to 5, preferably in position −20 or position 1, and ending with an amino acid in position 652 (GHPO 1180);
in SEQ ID NO:12, beginning with an amino acid in any one of positions −18 to 5, preferably in position −18 or position 1, and ending with an amino acid in position 673 (GHPO 896);
in SEQ ID NO:14, beginning with an amino acid in any one of positions −21 to 5, preferably in position −21 or position 1, and ending with an amino acid in position 619 (GHPO 711);
in SEQ ID NO:16, beginning with an amino acid in any one of positions −17 to 5, preferably in position −17 or position 1, and ending with an amino acid in position 635 (GHPO 190);
in SEQ ID NO:18, beginning with an amino acid in any one of positions −19 to 5, preferably in position −19 or position 1, and ending with an amino acid in position 626 (GHPO 185);
in SEQ ID NO:20, beginning with an amino acid in any one of positions −16 to 5, preferably in position −16 or position 1, and ending with an amino acid in position 467 (GHPO 1417); and
in SEQ ID NO:22, beginning with an amino acid in any one of positions −18 to 5, preferably in position −18 or position 1, and ending with an amino acid in position 673 (GHPO 1414); or
(ii) a derivative of the polypeptide.

2. An isolated polynucleotide that encodes:
(i) a polypeptide comprising an amino acid sequence that is homologous to an amino acid sequence selected from the group consisting of the amino acid sequences as shown:
in SEQ ID NO:2, beginning with amino acid in position −19 and ending with an amino acid in position 689 (GHPO 386);
in SEQ ID NO:4, beginning with an amino acid in position −20 and ending with an amino acid in position 713 (GHPO 789);
in SEQ ID NO:6, beginning with an amino acid in position −20 and ending with an amino acid in position 725 (GHPO 1516);
in SEQ ID NO:8, beginning with an amino acid in position −20 and ending with an amino acid in position 691 (GHPO 1197);
in SEQ ID NO:10, beginning with an amino acid in position −20 and ending with an amino acid in position 652 (GHPO 1180);
in SEQ ID NO:12, beginning with an amino acid in position −18 and ending with an amino acid in position 673 (GHPO 896);
in SEQ ID NO:14, beginning with an amino acid in position −21 and ending with an amino acid in position 619 (GHPO 711);
in SEQ ID NO:16, beginning with an amino acid in position −17 and ending with an amino acid in position 635 (GHPO 190);
in SEQ ID NO:18, beginning with an amino acid in position −19 and ending with an amino acid in position 626 (GHPO 185);
in SEQ ID NO:20, beginning with an amino acid in position −16 and ending with an amino acid in position 467 (GHPO 1417); and
in SEQ ID NO:22, beginning with an amino acid in position −18 and ending with an amino acid in position 673 (GHPO 1414); or
(ii) a derivative of the polypeptide.

3. The isolated polynucleotide of claim 1, which encodes the mature form of:
(i) a polypeptide comprising an amino acid sequence that is homologous to an amino acid sequence selected from the group consisting of the amino acid sequences as shown:
in SEQ ID NO:2, beginning with an amino acid in any one of positions −19 to 5, preferably in position −19 or position 1, and ending with an amino acid in position 689 (GHPO 386);
in SEQ ID NO:4, beginning with an amino acid in any one of positions −20 to 5, preferably in position −20 or position 1, and ending with an amino acid in position 713 (GHPO 789);
in SEQ ID NO:6, beginning with an amino acid in any one of positions −20 to 5, preferably in position −20 or position 1, and ending with an amino acid in position 725 (GHPO 1516);
in SEQ ID NO:8, beginning with an amino acid in any one of positions −20 to 5, preferably in position −20 or position 1, and ending with an amino acid in position 691 (GHPO 1197);
in SEQ ID NO:10, beginning with an amino acid in any one of positions −20 to 5, preferably in position −20 or position 1, and ending with an amino acid in position 652 (GHPO 1180);
in SEQ ID NO:12, beginning with an amino acid in any one of positions −18 to 5, preferably in position −18 or position 1, and ending with an amino acid in position 673 (GHPO 896);
in SEQ ID NO:14, beginning with an amino acid in any one of positions −21 to 5, preferably in position −21 or position 1, and ending with an amino acid in position 619 (GHPO 711);
in SEQ ID NO:16, beginning with an amino acid in any one of positions −17 to 5, preferably in position −17 or position 1, and ending with an amino acid in position 635 (GHPO 190);
in SEQ ID NO:18, beginning with an amino acid in any one of positions −19 to 5, preferably in position −19 or position 1, and ending with an amino acid in position 626 (GHPO 185);
in SEQ ID NO:20, beginning with an amino acid in any one of positions −16 to 5, preferably in position −16 or position 1, and ending with an amino acid in position 467 (GHPO 1417); and
in SEQ ID NO:22, beginning with an amino acid in any one of positions −18 to 5, preferably in position −18 or position 1, and ending with an amino acid in position 673 (GHPO 1414); or
(ii) a derivative of the polypeptide.

4. The isolated polynucleotide of claim 1, 2, or 3, wherein the polynucleotide is a DNA molecule.

5. The isolated polynucleotide of claim 1, which is a DNA molecule that can be amplified and/or cloned by polymerase chain reaction from an Helicobacter genome, using either:
a 5′ oligonucleotide primer having a sequence as shown in SEQ ID NO:23, and a 3′ oligonucleotide primer having a sequence as shown in SEQ ID NO:25 (unprocessed GHPO 386);
a 5′ oligonucleotide primer having a sequence as shown in SEQ ID NO:26, and a 3′ oligonucleotide primer having a sequence as shown in SEQ ID NO:28 (unprocessed GHPO 789);
a 5′ oligonucleotide primer having a sequence as shown in SEQ ID NO:29, and a 3′ oligonucleotide primer having a sequence as shown in SEQ ID NO:31 (unprocessed GHPO 1516);
a 5′ oligonucleotide primer having a sequence as shown in SEQ ID NO:32, and a 3′ oligonucleotide primer having a sequence as shown in SEQ ID NO:34 (unprocessed GHPO 1197);
a 5′ oligonucleotide primer having a sequence as shown in SEQ ID NO:35, and a 3′ oligonucleotide primer having a sequence as shown in SEQ ID NO:37 (unprocessed GHPO 1180);
a 5′ oligonucleotide primer having a sequence as shown in SEQ ID NO:38, and a 3′ oligonucleotide primer having a sequence as shown in SEQ ID NO:40 (unprocessed GHPO 896);
a 5′ oligonucleotide primer having a sequence as shown in SEQ ID NO:41, and a 3′ oligonucleotide primer having a sequence as shown in SEQ ID NO:43 (unprocessed GHPO 711);
a 5′ oligonucleotide primer having a sequence as shown in SEQ ID NO:44, and a 3′ oligonucleotide primer having a sequence as shown in SEQ ID NO:46 (unprocessed GHPO 190);
a 5′ oligonucleotide primer having a sequence as shown in SEQ ID NO:47, and a 3′ oligonucleotide primer having a sequence as shown in SEQ ID NO:49 (unprocessed GHPO 185);
a 5′ oligonucleotide primer having a sequence as shown in SEQ ID NO:50, and a 3′ oligonucleotide primer having a sequence as shown in SEQ ID NO:52 (unprocessed GHPO 1417);
a 5′ oligonucleotide primer having a sequence as shown in SEQ ID NO:53, and a 3′ oligonucleotide primer having a sequence as shown in SEQ ID NO:55 (unprocessed GHPO 1414);
a 5′ oligonucleotide primer having a sequence as shown in SEQ ID NO:24, and a 3′ oligonucleotide primer having a sequence as shown in SEQ ID NO:25 (mature GHPO 386);
a 5′ oligonucleotide primer having a sequence as shown in SEQ ID NO:27, and a 3′ oligonucleotide primer having a sequence as shown in SEQ ID NO:28 (mature GHPO 789);
a 5′ oligonucleotide primer having a sequence as shown in SEQ ID NO:30, and a 3′ oligonucleotide primer having a sequence as shown in SEQ ID NO:31 (mature GHPO 1516);
a 5′oligonucleotide primer having a sequence as shown in SEQ ID NO:33, and a 3′ oligonucleotide primer having a sequence as shown in SEQ ID NO:34 (mature GHPO 1197);
a 5′ oligonucleotide primer having a sequence as shown in SEQ ID NO:36, and a 3′ oligonucleotide primer having a sequence as shown in SEQ ID NO:37 (mature GHPO 1180);
a 5′oligonucleotide primer having a sequence as shown in SEQ ID NO:39, and a 3′ oligonucleotide primer having a sequence as shown in SEQ ID NO:40 (mature GHPO 896);
a 5′ oligonucleotide primer having a sequence as shown in SEQ ID NO:42, and a 3′ oligonucleotide primer having a sequence as shown in SEQ ID NO:43 (mature GHPO 711);
a 5′ oligonucleotide primer having a sequence as shown in SEQ ID NO:45, and a 3′ oligonucleotide primer having a sequence as shown in SEQ ID NO:46 (mature GHPO 190);
a 5′ oligonucleotide primer having a sequence as shown in SEQ ID NO:48, and a 3′ oligonucleotide primer having a sequence as shown in SEQ ID NO:49 (mature GHPO 185);
a 5′ oligonucleotide primer having a sequence as shown in SEQ ID NO:51, and a 3′ oligonucleotide primer having a sequence as shown in SEQ ID NO:52 (mature GHPO 1417); or
a 5′ oligonucleotide primer having a sequence as shown in SEQ ID NO:54, and a 3′ oligonucleotide primer having a sequence as shown in SEQ ID NO:55 (mature GHPO 1414).

6. The isolated DNA molecule of claim 5, which can be amplified and/or cloned by the polymerase chain reaction from a Helicobacter pylori genome.

7. The isolated polynucleotide of claim 1, which is a DNA molecule that encodes the mature form or a derivative of a polypeptide encoded by the DNA molecule of claim 5.

8. The isolated polynucleotide of claim 1, which is a DNA molecule that encodes the mature form or a derivative of a polypeptide encoded by the DNA molecule of claim 6.

9. A compound, in a substantially purified form, that is the mature form or a derivative of a polypeptide comprising an amino acid sequence that is homologous to an amino acid sequence of a polypeptide associated with the Helicobacter membrane, which is selected from the group consisting of the amino acid sequences as shown:
in SEQ ID NO:2, beginning with amino acid in position −19 and ending with an amino acid in position 689 (GHPO 386);
in SEQ ID NO:4, beginning with an amino acid in position −20 and ending with an amino acid in position 713 (GHPO 789);
in SEQ ID NO:6, beginning with an amino acid in position −20 and ending with an amino acid in position 725 (GHPO 1516);
in SEQ ID NO:8, beginning with an amino acid in position −20 and ending with an amino acid in position 691 (GHPO 1197);
in SEQ ID NO:10, beginning with an amino acid in position −20 and ending with an amino acid in position 652 (GHPO 1180);
in SEQ ID NO:12, beginning with an amino acid in position −18 and ending with an amino acid in position 673 (GHPO 896);
in SEQ ID NO:14, beginning with an amino acid in position −21 and ending with an amino acid in position 619 (GHPO 711);
in SEQ ID NO:16, beginning with an amino acid in position −17 and ending with an amino acid in position 635 (GHPO 190);
in SEQ ID NO:18, beginning with an amino acid in position −19 and ending with an amino acid in position 626 (GHPO 185);
in SEQ ID NO:20, beginning with an amino acid in position −16 and ending with an amino acid in position 467 (GHPO 1417); and
in SEQ ID NO:22, beginning with an amino acid in position −18 and ending with an amino acid in position 673 (GHPO 1414); or
(ii) a derivative of the polypeptide.

10. The compound of claim 9, which is the mature form or a derivative of a polypeptide encoded by a DNA molecule of claim 5.

11. The compound of claim 9, which is the mature form or a derivative of a polypeptide encoded by a DNA molecule of claim 6.

12. A method of preventing or treating Helicobacter infection in a mammal, said method comprising administering to said mammal a prophylactically or therapeutically effective amount of a compound of claim 9, 10, or 11.

13. The method of claim 12, further comprising administering an antibiotic, an antisecretory agent, a bismuth salt, or a combination thereof.

14. The method of claim 13, wherein said antibiotic is selected from the group consisting of amoxicillin, clarithromycin, tetracycline, metronidizole, and erythromycin.

15. The method of claim 13, wherein said bismuth salt is selected from the group consisting of bismuth subcitrate and bismuth subsalicylate.

16. The method of claim 13, wherein said antisecretory agent is a proton pump inhibitor.

17. The method of claim 16, wherein said proton pump inhibitor is selected from the group consisting of omeprazole, lansoprazole, and pantoprazole.

18. The method of claim 13, wherein said antisecretory agent is an H₂-receptor antagonist.

19. The method of claim 18, wherein said H₂-receptor antagonist is selected from the group consisting of ranitidine, cimetidine, famotidine, nizatidine, and roxatidine.

20. The method of claim 13, wherein said antisecretory agent is a prostaglandin analog.

21. The method of claim 20, wherein said prostaglandin analog is misoprostil or enprostil.

22. The method of claim 12, which further comprises administering a prophylactically or therapeutically effective amount of a second Helicobacter polypeptide or a derivative thereof.

23. The method of claim 22, wherein the second Helicobacter polypeptide is a Helicobacter urease, a subunit, or a derivative thereof.

24. A composition comprising a compound of claim 9, 10, or 11, together with a physiologically acceptable diluent or carrier.

25. The composition of claim 24, further comprising an adjuvant.

26. The composition of claim 24, further comprising a second Helicobacter polypeptide or a derivative thereof.

27. The composition of claim 26, wherein said second Helicobacter polypeptide is a Helicobacter urease, or a subunit or a derivative thereof.

28. A method of preventing or treating Helicobacter infection in a mammal, said method comprising administering to said mammal a prophylactically or therapeutically effective amount of a polynucleotide of claim 1, 2, or 3.

29. A method of preventing or treating Helicobacter infection in a mammal, said method comprising administering to said mammal a prophylactically or therapeutically effective amount of a polynucleotide of claim 5, 6, or 7.

30. A method of preventing or treating Helicobacter infection in a mammal, said method comprising administering to said mammal a prophylactically or therapeutically effective amount of a polynucleotide of claim 8.

31. A composition comprising a viral vector, in the genome of which is inserted a DNA molecule of claim 4, said DNA molecule being placed under conditions for expression in a mammalian cell and said viral vector being admixed with a physiologically acceptable diluent or carrier.

32. The composition of claim 31, wherein said viral vector is a poxvirus.

33. A composition that comprises a bacterial vector comprising a DNA molecule of claim 4, said DNA molecule being placed under conditions for expression and said bacterial vector being admixed with a physiologically acceptable diluent or carrier.

34. The composition of claim 33, wherein said vector is selected from the group consisting of Shigella, Salmonella, Vibrio cholerae, Lactobacillus, Bacille bilié de Calmette-Guérin, and Streptococcus.

35. A composition comprising a polynucleotide of claim 1, 2, or 3, together with a physiologically acceptable diluent or carrier.

36. The composition of claim 35, wherein said polynucleotide is a DNA molecule that is inserted in a plasmid that is unable to replicate and to substantially integrate in a mammalian genome and is placed under conditions for expression in a mammalian cell.

37. An expression cassette comprising a DNA molecule of claim 4, said DNA molecule being placed under conditions for expression in a procaryotic or eucaryotic cell.

38. A process for producing a compound of claim 9, which comprises culturing a procaryotic or eucaryotic cell transformed or transfected with an expression cassette of claim 37, and recovering said compound from the cell culture.

39. A method of preventing or treating Helicobacter infection in a mammal, said method comprising administering to said mammal a prophylactically or therapeutically effective amount of an antibody that binds to the compound of claim 9, 10, or 11.


SEQUENCE LISTING (64 sequences)

SEQ ID NO: 1 - DNA (Genomic DNA), 2798 bp; single-stranded, linear
  Coding Sequence 328...2451: (A) NAME/KEY Signal Sequence (B) LOCATION 328...385 (D) OTHER INFORMATION
tggtcctggcattccgaggttcgaatccttgcaccccagccatttttccttattttttggcgcggagtagagcagtccggtagctcgttgggctcataacccaaaggtcagtggttcaaatccattctccgcaaccaatcctttaaaccacaccaccaccaaacgaaccaaacgaaacaaaaagcatcaaaatcaaaaaaatgacaaaatttttaagaaaatgacaaaaaaaaaaaaaacgattttatgctatattaacgaaatcttgtgataagatcttattcttttaaaagacttatctaaccattttaatttcaaggagaaaacatgaaaaaaacccttttactctctctctctctctctctctcgtttttgctccacgctgaagacgacggcttttacacaagcgtgggctatcaaatcggtgaagccgctcaaatggtgaaaaacaccaaaggcattcaagagctttcagacaattatgaaaagctgaacaatcttttgaataattacagcaccctaaacacccttatcaaattgtccgctgatccgagcgcgattaacgacgcaagggataatctaggctcaagctctaggaatttgcttgatgtcaaaaccaattcccccgcgtatcaagccgtgcttttagcactcaatgctgcagtggggttgtggcaagttacaagctacgcttttactgcttgtggtcctggcagtaacgagaatgcgaatggagggatccaaacttttaataatgtgccaggacaagatacgacgaccatcacttgcaattcgtattatgagccaggacatggtgggcctatatccactgcaaattatgcgaaaatcaatcaagcctatcaaatcatccaaaaggctttgacagccaatggagctaatggagatggggtccccgttttaagcaacaccactacaaaacttgatttcactatcaatggagacaaaagaacggggggcaaaccaaatacacctgaaaagttcccatggagtgatgggaaatatattcacacccaatggattaacacaatagtaacaccaacagaaacaaatatcaacacagaaaataacgctcaagagcttttaaaacaagcgagcatcattatcactaccctaaatgaggcatgcccaaacttccaaaatggtggtagaagttattggcaagggataagcggcaatgggacaatgtgcgggatgtttaagaatgaaatcagcgcgatccaaggcatgatcgctaacgctcaagaagctgtcgcgcaaagcaaaatcgttagtgaaaacgcgcaaaatcaaaacaacttggatactggaaaaccattcaacccttacacggacgccagctttgcgcaaagcatgctcaaaaacgctcaagcgcaagcagagattttaaaccaagccgaacaagtagtaaaaaactttgaaaaaatccctacagcctttgtatcagactctttaggggtgtgttatgaagtgcaagggggtgagcgtaggggcaccaatccaggtcaggtaacttctaacacttggggagccggttgcgcgtatgtgaaacaaaccataacgaatttagacaacagcatcgctcactttggcactcaagagcagcagatacagcaagccgaaaacatcgctgacactctagtgaatttcaaatctagatacagcgaattaggcaacacctataacagcatcaccaccgcgctctccaaagtccctaacgcgcaaagcttgcaaaacgtggtgagcaaaaagaataacccctatagccctcaaggcatagagaccaattactacctcaatcaaaattcttacaaccaaatccaaaccatcaaccaagaactagggcgtaacccctttaggaaagtgggcatcgtcaattctcaaaccaacaatggtgccatgaatgggatcggtattcaggtgggctataagcaattctttggccaaaaaagaaaatggggcgctaggtattacggcttttttgactacaaccatgcgttcattaaatccagcttcttcaactcggcttctgatgtgtggacttatggttttggagcggacgctctttataacttcatcaacgataaagccaccaatttcttaggcaaaaacaacaagctttccgtggggctttttggagggattgcgttagcgggcacttcatggcttaattctgagtatgtgaatttagccaccgtgaataacgtctataacgctaaaatgaatgtggcgaatttccaattcttattcaatatgggagtgaggatgaatttagccagatccaagaaaaaaggcagcgatcatgcggctcagcatgggattgaactagggcttaaaatccccaccatcaacacgaactattattctttcatgggggctgaactcaaatacagaaggctttatagcgtgtatttgaattatgtgttcgcttactaagctttttgtgaaactccctttttaaggggtttttttttgaactctctttttaaattctctttttaaagagatttcttttttttaagcttttttttgaattcttttttttgaattctttgtttttaagctttttttaaaccctttcgtttttaaactcccttttttaagggatttctttttttaaactctttttttttaaactcttttttttaaaccctctttttttaagggatttctttttaaagcttttttgaagtctttttttaaattctttttttgggggtttgatctttctttttgccaatccccactactttcgctttttaatctttaggttttatttt

SEQ ID NO: 2 - protein, 708 aa
  Signal Sequence 1...19
  fragment type: internal
MKKTLLLSLSLSLSFLLHAEDDGFYTSVGYQIGEAAQMVKNTKGIQELSDNYEKLNNLLNNYSTLNTLIKLSADPSAINDARDNLGSSSRNLLDVKTNSPAYQAVLLALNAAVGLWQVTSYAFTACGPGSNENANGGIQTFNNVPGQDTTTITCNSYYEPGHGGPISTANYAKINQAYQIIQKALTANGANGDGVPVLSNTTTKLDFTINGDKRTGGKPNTPEKFPWSDGKYIHTQWINTIVTPTETNINTENNAQELLKQASIIITTLNEACPNFQNGGRSYWQGISGNGTMCGMFKNEISAIQGMIANAQEAVAQSKIVSENAQNQNNLDTGKPFNPYTDASFAQSMLKNAQAQAEILNQAEQVVKNFEKIPTAFVSDSLGVCYEVQGGERRGTNPGQVTSNTWGAGCAYVKQTITNLDNSIAHFGTQEQQIQQAENIADTLVNFKSRYSELGNTYNSITTALSKVPNAQSLQNVVSKKNNPYSPQGIETNYYLNQNSYNQIQTINQELGRNPFRKVGIVNSQTNNGAMNGIGIQVGYKQFFGQKRKWGARYYGFFDYNHAFIKSSFFNSASDVWTYGFGADALYNFINDKATNFLGKNNKLSVGLFGGIALAGTSWLNSEYVNLATVNNVYNAKMNVANFQFLFNMGVRMNLARSKKKGSDHAAQHGIELGLKIPTINTNYYSFMGAELKYRRLYSVYLNYVFAY

SEQ ID NO: 3 - DNA (Genomic DNA), 2699 bp; single-stranded, linear
  Coding Sequence 199...2397: (A) NAME/KEY Signal Sequence (B) LOCATION 199...259 (D) OTHER INFORMATION
taaaatccaattaaaagcgttcaaaggtaacgcaaaaaaacaaaaaatgacgcaattttttcaaaatgacaaaaaaaaacgctttatgctataataccccaaatacattctaatagcaaatgcgttctaatgcaaatgcattccaatgtatgaaatccctaatactaaatccaatttaatccaaaaaggagaaaaaacatgaaaaaacacatcctttcattagctttaggctcgcttttagtttccactttgagcgctgaagacgacggcttttacacaagcgtaggctatcagatcggtgaagccgctcaaatggtaacaaacaccaaaggcatccaacagctttcagacaattatgaaaatttgaacaaccttttaacgagatacagcaccctaaacacccttatcaaattgtccgctgatccgagcgcaattaatgcggtgcgggaaaatctgggcgcgagcgcgaagaatttgatcggcgataaagccaactcccccgcctatcaagccgtgcttttagcgatcaacgcggcggtagggttttggaatgtcgtgggctatgtgacgcaatgtgggggtaacgccaatggtcaagaaagcacctcttcaaccaccatcttcaacaacgagccagggtatcgatccacttccatcacttgttctttgaacgggcataagcctggatactatggccctatgagcattgagaattttaaaaagcttaacgaagcctatcagatcctccaaacggctttaaaaaacggcttacccgcgctcaaagaaaacaacgggaaggtcagtgtaacctatacctacacatgctcagggcaagggaataataactgctcgccaagtgtcaacggaaccaaaaccacaacccaaaccatagacggcaaaagcgtaaccaccacgatcagttcaaaagtggttggtagcatcgctagtggcaacacatcacatgtcatcaccaacaaattagacggtgtgcctgatagcgctcaagcgctcttagcgcaagcgagcacgctcatcaacaccatcaacgaagcatgcccgtatttccatgctactaatagtagtgaggctaacgccccaaaattctctactactactgggaaaatatgcggcgctttttcagaagaaatcagcgcgatccaaaagatgatcacggacgcgcaagagctagttaatcaaacgagcgtcattaacagcaacgaacaatcaactccggtaggcaataataatggcaagcctttcaaccctttcacggacgcaagttttgcgcaaggcatgctcgctaacgctagcgcgcaagctaaaatgctcaatttagcccatcaggtggggcaagccattaacccagagaatcttagcgagaattttaaaaattttgttacaggctttttagccacatgcaataacaaatcaacagctggcactggtggcacacaaggttcagctccaggcacagtgaccactcaaactttcgcttctggttgcgcgtatgtggagcaaaccctaacgaacttaggcaacagcatcgctcactttggcactcaagagcagcagatacagcaagccgaaaacatcgctgacactctagtgaatttcaaatctagatacagcgaattaggcaacacctataacagcatcaccaccgcgctctccaaagtccctaacgcgcaaagcttgcaaaacgtggtgagcaaaaagaataacccctatagccctcaaggcatagagaccaattactacctcaatcaaaattcttacaaccaaatccaaaccatcaaccaagaactagggcgtaacccctttaggaaagtgggcatcgtcaattctcaaaccaacaatggtgccatgaatgggatcggtattcaggtgggctataagcaattctttggccaaaaaagaaaatggggcgctaggtattacggcttttttgattacaaccatgcgttcatcaaatccagctttttcaactcggcttctgacgtgtggacttatggttttggagcggacgcgctttataacttcatcaacgataaagccaccaatttcttaggcaaaaacaacaagctttctttggggctttttggcgggattgcgttagcgggcacttcatggctcaattctgagtacgtgaatttagccaccgtgaataacgtctataacgctaaaatgaatgtggcgaatttccaattcttattcaatatgggagtgaggatgaatttagccagatccaagaaaaaaggcagcgatcatgcagctcagcatgggattgagttagggcttaaaatccccaccatcaacacgaactattattcctttatgggggctgaactcaaatacagaaggctctatagcgtgtatttgaactatgtgttcgcttactaatgtttggctctttgtgaaactccctttttaaggggtttttttttgaactctctttttaaattctctttttaaagagatttcttttttttaagcttttttttgaattctttttttttgaattctttgtttttaagctttttttaaaccctttcgtttttaaactcccttttttaagggatttctttttttgaactcccttttttgaacccttttttttaaaccctctttttttaaggggtttctttttaaagcttttttgaagtctttttttaaattctttttttgggggtttgatcttt

SEQ ID NO: 4 - protein, 733 aa
  Signal Sequence 1...20
  fragment type: internal
MKKHILSLALGSLLVSTLSAEDDGFYTSVGYQIGEAAQMVTNTKGIQQLSDNYENLNNLLTRYSTLNTLIKLSADPSAINAVRENLGASAKNLIGDKANSPAYQAVLLAINAAVGFWNVVGYVTQCGGNANGQESTSSTTIFNNEPGYRSTSITCSLNGHKPGYYGPMSIENFKKLNEAYQILQTALKNGLPALKENNGKVSVTYTYTCSGQGNNNCSPSVNGTKTTTQTIDGKSVTTTISSKVVGSIASGNTSHVITNKLDGVPDSAQALLAQASTLINTINEACPYFHATNSSEANAPKFSTTTGKICGAFSEEISAIQKMITDAQELVNQTSVINSNEQSTPVGNNNGKPFNPFTDASFAQGMLANASAQAKMLNLAHQVGQAINPENLSENFKNFVTGFLATCNNKSTAGTGGTQGSAPGTVTTQTFASGCAYVEQTLTNLGNSIAHFGTQEQQIQQAENIADTLVNFKSRYSELGNTYNSITTALSKVPNAQSLQNVVSKKNNPYSPQGIETNYYLNQNSYNQIQTINQELGRNPFRKVGIVNSQTNNGAMNGIGIQVGYKQFFGQKRKWGARYYGFFDYNHAFIKSSFFNSASDVWTYGFGADALYNFINDKATNFLGKNNKLSLGLFGGIALAGTSWLNSEYVNLATVNNVYNAKMNVANFQFLFNMGVRMNLARSKKKGSDHAAQHGIELGLKIPTINTNYYSFMGAELKYRRLYSVYLNYVFAY

SEQ ID NO: 5 - DNA (Genomic DNA), 2915 bp; single-stranded, linear
  Coding Sequence 365...2597: (A) NAME/KEY Signal Sequence (B) LOCATION 365...425 (D) OTHER INFORMATION
ttttaggcgacaaaatcgcttatgttggggataaaggcaacccgcacaatttcgctcacaagaaataaaccgctcataaggggcaaacgccccaaaaaagcgatttttaaagaggttacggcaaaatcaagctctttagtatttaatcttaaaaaatgctaaaagcctttttatgggctaacaccacacaaaaagcatcaaaatcaaaaaaatgacaaaatttttaagaaaatgacaaaaaaaaacgctttatgctataataccccaaatacattctaatagcaaatgcgttctaatgcaaatgcattccaatgtatgaaatccctaatactaaatccaatttaatccaaaaaggagaaaaaacatgaaaaaacacatcctttcattagctttaggctcgcttttagtttccactttgagcgctgaagacgacggcttttacacaagcgtaggctatcagatcggtgaagccgctcaaatggtaacaaacaccaaaggcatccaacagctttcagacaattatgaaaatttgaacaaccttttaacgagatacagcaccctaaacacccttatcaaattgtccgctgatccgagcgcaattaatgcggtgcgggaaaatctgggcgcgagcacgaagaatttgatcggcgataaagccaactccccggcgtatcaagccgtgtttttagcgatcaacgcggcggtagggttgtggaataccatcggctatgcggtcatgtgcgggaacgggaacggcacagagagtgggcctggcagcgtgatctttaatgaccaaccaggacaggattccacgcaaattacttgcaaccgctttgaatcaactgggcctggtaaaagcatgtctattgatgaattcaaaaaactcaatgaagcctatcaaatcatccagcaagctttaaaaaatcaaagtgggtttcctgaattaggcgggaacggcacaaaagtgagtgttaattacaattacgaatgcagacaaactgctgatatcaacggcggtgtgtatcagttctgcaaggctaaaaatggtagtagtagcagtagtaatggcggtaatggcagtagcacgcaaacaaccgcgacaaccacgcaagacggcgtaacgatcaccactacctataataataacaaagccaccgtcaaatttgacatcaccaataacgctgaacagctgttaaatcaagcggcaaacatcatgcaagtccttaatacgcaatgccctttagtgcgttccacgaataacgaaaacactccagggggtggtcaaccatggggtttaagcacatccgggaatgcgtgcagcatcttccaacaagaatttagccaggttactagcatgatcaaaaacgcccaagaaataatcgcgcaaagcaaaatcgttagtgaaaacgcgcaaaatcaaaacaacttggatactggaaaaccattcaacccttacacggacgccagctttgcgcaaagcatgctcaaaaacgctcaagcgcaagcagagatgttcaatttgagcgaacaagtgaaaaagaacttggaagtcatgaaaaacaacaataatgttaacgagaaattagcaggatttgggaaagaagaagtaatgaccaattttgttagcgcctttttggcaagctgcaaagatggtggcacattgcctaatgcaggggttacttctaacacttggggggcgggttgcgcgtatgtgggagagacgataagcgccctaaccaacagcatcgctcactttggcactcaagagcagcagatacagcaagccgaaaacatcgctgacactctagtgaatttcaaatctagatacagcgaattaggcaacacctataacagcatcaccaccgcgctctccaaagtccctaacgcgcaaagcttgcaaaacgtggtgagcaaaaagaataacccctatagccctcaaggcatagagaccaattactacctcaatcaaaattcttacaaccaaatccaaaccatcaaccaagaactagggcgtaacccctttaggaaagtgggcatcgtcaattctcaaaccaacaatggtgccatgaatgggatcggcattcaggtgggctataagcaattctttggccaaaaaagaaaatggggcgctaggtattacggcttttttgattacaaccatgcgttcatcaaatccagctttttcaactcggcttctgacgtgtggacttatggttttggagcggacgcgctttataacttcatcaacgataaagccaccaatttcttaggcaaaaacaacaagctttctttggggctttttggcgggattgcgttagcgggcacttcatggctcaattctgagtacgtgaatttagccaccgtgaataacgtctataacgctaaaatgaatgtggcgaatttccaattcttattcaatatgggagtgaggatgaatttagccagatccaagaaaaaaggcagcgatcatgcagctcagcatgggattgagttagggcttaaaatccccaccatcaacacgaactattattcctttatgggggctgaactcaaatacagaaggctctatagcgtgtatttgaatnatgtgttcgcttactaagctttttgtgaaactccctttttaaggggtttttttttgaactctcttttaaattctctttttaaagagatttctttttttaagcttttttttgaacttttttttgaattctttgtttttaagctttttttaaaccctttcgtttttaaactcccttttttaagggatttctttttttgaactcccttttttgaacccttttttttaaaccctctttttttaaggggtttctttttaaagcttttttgaagtctttttttaaattctttttttgggggtttgatctttctttttgccaatccccactactttc

SEQ ID NO: 6 - protein, 745 aa
  Signal Sequence 1...20
  fragment type: internal
MKKHILSLALGSLLVSTLSAEDDGFYTSVGYQIGEAAQMVTNTKGIQQLSDNYENLNNLLTRYSTLNTLIKLSADPSAINAVRENLGASTKNLIGDKANSPAYQAVFLAINAAVGLWNTIGYAVMCGNGNGTESGPGSVIFNDQPGQDSTQITCNRFESTGPGKSMSIDEFKKLNEAYQIIQQALKNQSGFPELGGNGTKVSVNYNYECRQTADINGGVYQFCKAKNGSSSSSNGGNGSSTQTTATTTQDGVTITTTYNNNKATVKFDITNNAEQLLNQAANIMQVLNTQCPLVRSTNNENTPGGGQPWGLSTSGNACSIFQQEFSQVTSMIKNAQEIIAQSKIVSENAQNQNNLDTGKPFNPYTDASFAQSMLKNAQAQAEMFNLSEQVKKNLEVMKNNNNVNEKLAGFGKEEVMTNFVSAFLASCKDGGTLPNAGVTSNTWGAGCAYVGETISALTNSIAHFGTQEQQIQQAENIADTLVNFKSRYSELGNTYNSITTALSKVPNAQSLQNVVSKKNNPYSPQGIETNYYLNQNSYNQIQTINQELGRNPFRKVGIVNSQTNNGAMNGIGIQVGYKQFFGQKRKWGARYYGFFDYNHAFIKSSFFNSASDVWTYGFGADALYNFINDKATNFLGKNNKLSLGLFGGIALAGTSWLNSEYVNLATVNNVYNAKMNVANFQFLFNMGVRMNLARSKKKGSDHAAQHGIELGLKIPTINTNYYSFMGAELKYRRLYSVYLNXVFAY

SEQ ID NO: 7 - DNA (Genomic DNA), 2603 bp; single-stranded, linear
  Coding Sequence 210...2342: (A) NAME/KEY Signal Sequence (B) LOCATION 210...270 (D) OTHER INFORMATION
atgacctttattggtttaatatttgtttagaaataacacaaaaacctttttttttttttttgaaagggcaaaaacgcctaattaatatcaaaatcccatgaatttatactatattaacgaaagcttgcggtatggtttcacctaaagacacacttccgcaagatttactaacaatttcaatcttatttcaagtaataaaaggagaaaacatgaagaaaaaatttctgtcattaaccttaggttcgcttttagtttccgctttaagcgctgaagacaacggcttttttgtgagtgcgggctatcaaatcggtgaatccgctcaaatggtgaaaaacactaaaggcattcaagatctttcagatagctatgaaagactgaacaatcttttaacgagttatagtgccctaaacactcttattaggcagtccgccgaccccaacgctatcaataacgcaaggggcaatttgaacgctagtgcgaagaatttgatcaatgataaaaagaattccccggcgtatcaagcggtgcttttagccttgaatgcggcagcggggttgtggcaagtcatgagctattcgatcagcgtttgtggccctggctctgacaaaaataaaaatgggggcgtccaaacctttgaaaatgtgccgtcaaatggggggactaccattgcttgcgattcattttatgaaccaggaaagtggagcggtatatccactgaaaattacgcaaaaatcaataaagcctatcaaatcatccaaaaggcttttggagcaagcgggcaagatattcctgccttaagcgacaccaaagaacttaattttgaaattaaagggaaaaaaaatgatagcgtccagccaggagaaagatggaaattcccatggactaatggaaaatttgtttcagtcaagtgggtgaatgggaagtatgaagaaattaaagaagacatcaaagtgtcaaataacgctcaagagcttttaaaacaggctagcactattttaaccactcttaatgaagcatgcccatggttgagtaatggtggtgcaggcaatgtggccggtggcaatagtttatgggccggaatagataaaggcgacgggagcgcatgcgggatttttaaaaatgaaatcagcgcgattcaagacatgatcaaaaacgctgaaatagccgtagagcaatccaaaatcgttaccgccaacgcgcaaaaccagcacaacctagacactgggaaagcattcaacccctataaagacgccaacttcgcccaaagcatgttcgctaacgctagagcgcaagcggagattttaaaccgcgctcaagcagtggtgaaggactttgaaagaatccctgcagcgttcgtgaaagactctttaggagtatgccatgaaaagggtagcgacggcaatctccgtggcacgccatctggcacggttacttctaacacttggggagccggctgcgcgtatgtgggagaaaccgtaacgaatctaaaaaacagcatcgctcattttggcgaccaagcggagcgaatccataatgcgcgaaatctcgcctacactttagcgaatttcagcggccagtacaaaaagctaggcgaacactatgacagcatcacagcggcgctctctagcttgcctgatgcgcaatctttacaaaatgtggtgagcaaaaagactaaccctaacagcccgcaaggcatacaggataattactacattgactccaacatccattctcaagtgcaatctaggagtcaagaactcggcagtaaccctttcagacgcgccgggctaatcgccgcttctaccaccaataacggcgcgatgaatgggattggctttcaagtgggctataagcaattctttgggaaaaacaaacgatggggcgcgagatactacggctttgtggattacaaccacacctataacaagtcccaatttttcaactccgattctgatgtttggacttatggcgtggggagcgatttgttagtgaatttcatcaacgataaagccactaaacacaataaaatttcttttggcgcgtttggcggtatccaactagccgggacttcatggcttaattctcagtatgtgaatttagcgaatgtgaacaattattataaagctaaaatcaacacctctaacttccaattcttattcaatctgggcttaaggaccaatctcgccagaaataaaagaataggcgctgatcatagcgcgcaacatggcatggaattaggcgtgaagatccccacgatcaacacaaattactattctttgctaggcactaccttgcaatacagaaggctttatagcgtgtatctcaactatgtgtttgcttactaaaagcttaaactcctttttaaactccctttttagggggtttaatctttttaactgacttttcttttagctttttttaattttttccaccaaacaaagttttttgacttcaagcgttaatcacaaaaaatactcaaaggcgttttttgcaatctaaataaaaaattagcgttattcaagcgatcattttaaaccacccaagcaagaaaccccaaacatctttagcgttcgcgcgctccactaaccaaaaaacgccccaaa

SEQ ID NO: 8 - protein, 711 aa
  Signal Sequence 1...20
  fragment type: internal
MKKKFLSLTLGSLLVSALSAEDNGFFVSAGYQIGESAQMVKNTKGIQDLSDSYERLNNLLTSYSALNTLIRQSADPNAINNARGNLNASAKNLINDKKNSPAYQAVLLALNAAAGLWQVMSYSISVCGPGSDKNKNGGVQTFENVPSNGGTTIACDSFYEPGKWSGISTENYAKINKAYQIIQKAFGASGQDIPALSDTKELNFEIKGKKNDSVQPGERWKFPWTNGKFVSVKWVNGKYEEIKEDIKVSNNAQELLKQASTILTTLNEACPWLSNGGAGNVAGGNSLWAGIDKGDGSACGIFKNEISAIQDMIKNAEIAVEQSKIVTANAQNQHNLDTGKAFNPYKDANFAQSMFANARAQAEILNRAQAVVKDFERIPAAFVKDSLGVCHEKGSDGNLRGTPSGTVTSNTWGAGCAYVGETVTNLKNSIAHFGDQAERIHNARNLAYTLANFSGQYKKLGEHYDSITAALSSLPDAQSLQNVVSKKTNPNSPQGIQDNYYIDSNIHSQVQSRSQELGSNPFRRAGLIAASTTNNGAMNGIGFQVGYKQFFGKNKRWGARYYGFVDYNHTYNKSQFFNSDSDVWTYGVGSDLLVNFINDKATKHNKISFGAFGGIQLAGTSWLNSQYVNLANVNNYYKAKINTSNFQFLFNLGLRTNLARNKRIGADHSAQHGMELGVKIPTINTNYYSLLGTTLQYRRLYSVYLNYVFAY

SEQ ID NO: 9 - DNA (Genomic DNA), 2427 bp; single-stranded, linear
  Coding Sequence 232...2247: (A) NAME/KEY Signal Sequence (B) LOCATION 232...292 (D) OTHER INFORMATION
aaaacgcgcagcaaaaaatctctgttaagcttttatcattagcgttccattgaaacaaaatctaaaaaccctttccaataccacccaaacaaacgcgcaaaaaatgcaaaaattctaaattttctccaaatgacaaaaaaaaaaaaaacgattttatgctacaatgcttttaatacattcttacttaatgtataaaatctcaatcactcaatttaatttcaaaggatatttatgaaaaaaacccttttactctctctctctctctctctctcgtcatcgcttttaaacgctgaagacaacggcttttttatcagcgcgggctatcaaatcggtgaagccgctcaaatggtgaaaaacaccggcgaattgaaaaaactttcagacacttatgagaatttgagcaaccttttaaccaattttaacaacctcaatcaagcggtaacgaacgcgagcagcccttcagaaatcaatgccacgatcgataatttaaaagcaaacacgcaagggctgattggcgaaaaaaccaattccccggcgtatcaagcggtgtatttggcgctcaatgcggcggtggggctgtggaatgtgatagcctataatgtccaatgcggtcctggtaagagtggggatcaaagcgtaatttttgatggccaaccaggacatgattcaagatccattaattgcaatttaaccggttataacaacggggttagcggccctttatccattgacaattttaaaacgcttaatcaagcttatcaaactatccaacaagctttaaaacaagatagcggatttcctgttttggatagtaaaggaaaacaagtaactataaaaataacaacacaaactaatggagctaataaaagtgaaactactactactactactactactaatgacgctcaaacccttttgcaagaagccagtaaaatgataagcgtcctcactacaaactgcccatgggtaaataccgctcataactcaaacgggggtgcaccgtggaatttaaatacgacagggaatgtgtgtcaggtttttgccacggagtttagcgccgttactagcatgatcaaaaacgcgcaagaaatcgtaacgcaagctcaaagccttaacaacccgcaaagcaatcaaaacgcgccgaaagatttcaatccttacacctctgctgatagggctttcgctcaaaacatgctcaatcacgcgcaagcgcaagccaagatgcttgaactagccgatcaaatgaaaaaagaccttaacactatcccaaaacaatttatcacaaactacttggcagcttgccgcaatgggggtgggacattacctgatgcaggggttacttctaacacttggggggccggttgcgcctatgtggaagagacgataaccgccctaaataacagccttgcgcattttggcactcaagccgatcaaatcaagcaatctgagttgttggcgcgcacgatacttgattttagaggcagccttaaggatttaaacaacacttataacagcatcaccacgaccgcttcaaacacgcccaattccccattccttaaaaatttgataagccaatccactaaccctaataaccccgggggcttacaggccgtttatcaagtcaaccaaagcgcttattcgcaattattaagcgccacgcaagaattagggcataaccctttcagacgcgttggcttaatcagctctcaaaccaacaacggtgcgatgaatgggatcggcgtgcaaatagggtataaacaattttttggtgaaaaaagaagatgggggttaaggtattatggtttttttgattacaaccatgcttatatcaaatccagctttttcaactccgcctctgatgtgttcacttatggggtaggaacagatgtcctctataactttatcaacgataaagccaccaaaaacaataagatttcttttggggtgtttggggggattgcgttagctggcacttcgtggcttaattctcaatacgtgaatttagcgacattcaataatttttacagcgctaaaatgaatgtggcgaatttccaattcttattcaacttgggcttgagaatgaatctcgctaaaaacaaaaagaaagcgagcgatcatgtagctcagcatggcgtggaactaggcgtgaagatccctacgatcaacacgaattactattctttgctaggcactcaactccaataccgcaggctttatagcgtgtatttgaattatgtgtttgcttactaatatctgtctttttgtgaaactccctttttaagggattttttttgaagcctttctttttttaaaccctcttttttgggggtcaagcgtaaaattcacccctatccctttaagaaaataaaataaaagaaaatgcgttttataacaaaataagatctaaaacaataaaacaaaaaccca

SEQ ID NO: 10 - protein, 672 aa
  Signal Sequence 1...20
  fragment type: internal
MKKTLLLSLSLSLSSSLLNAEDNGFFISAGYQIGEAAQMVKNTGELKKLSDTYENLSNLLTNFNNLNQAVTNASSPSEINATIDNLKANTQGLIGEKTNSPAYQAVYLALNAAVGLWNVIAYNVQCGPGKSGDQSVIFDGQPGHDSRSINCNLTGYNNGVSGPLSIDNFKTLNQAYQTIQQALKQDSGFPVLDSKGKQVTIKITTQTNGANKSETTTTTTTTNDAQTLLQEASKMISVLTTNCPWVNTAHNSNGGAPWNLNTTGNVCQVFATEFSAVTSMIKNAQEIVTQAQSLNNPQSNQNAPKDFNPYTSADRAFAQNMLNHAQAQAKMLELADQMKKDLNTIPKQFITNYLAACRNGGGTLPDAGVTSNTWGAGCAYVEETITALNNSLAHFGTQADQIKQSELLARTILDFRGSLKDLNNTYNSITTTASNTPNSPFLKNLISQSTNPNNPGGLQAVYQVNQSAYSQLLSATQELGHNPFRRVGLISSQTNNGAMNGIGVQIGYKQFFGEKRRWGLRYYGFFDYNHAYIKSSFFNSASDVFTYGVGTDVLYNFINDKATKNNKISFGVFGGIALAGTSWLNSQYVNLATFNNFYSAKMNVANFQFLFNLGLRMNLAKNKKKASDHVAQHGVELGVKIPTINTNYYSLLGTQLQYRRLYSVYLNYVFAY

SEQ ID NO: 11 - DNA (Genomic DNA), 2429 bp; single-stranded, linear
  Coding Sequence 205...2277: (A) NAME/KEY Signal Sequence (B) LOCATION 205...259 (D) OTHER INFORMATION
tgaaagaagactgattagtctttcttttaggggcgattcaagccttaaaagccgggtcaaaatccccatttttcccaatttttacaaaaaaaaaaaaaacaaaatctctaaaatttagagctaaaattagccataaaattccatttattgcttataatatgaagtttctttgtatcaaagaaaaatctattaaaaggagaaaacatgaaaaaatccctcttactctctctttctctcatcgcttccttatcaagagctgaagatgacggattttatacgagtgtgggctatcagatcggtgaagcggtccaacaagtgaaaaacacaggagcattgcaaaatcttgcagacagatacgataacttaaacaaccttttaaaccaatacaattatttaaattccttagtcaatttagccagcacgccgagcgcgatcaccggtgcgattgataatttaagctcaagcgcgattaacctcactagcgccaccaccacttcccccgcctatcaagctgtggctttagcgctcaatgccgctgtgggcatgtggcaagtcatagccctttttattggctgtggccctggccctaccaataatcaaagctatcaatcgtttggtaacacaccagcccttaatgggaccaccaccacttgcaatcaagcatatgggacaggccctaatggcatcctatctattgatgaataccaaaaactcaaccaagcttatcagatcatccaaaccgctttaaaccaaaatcaagggggtgggatgcctgccttgaatgacaccaccaaaacaggggtagtcaacatacaacaaaccaattataggaccaccacacaaaacaatatcatagagcattattatacagagaatgggaaagagatcccagtctcttattcaggcggatcatcattctcgcctacaatacaattgacataccataataacgctgaaaaccttttgcaacaagccgccactatcatgcaagtccttattactcaaaagccgcatgtgcaaacgagcaatggcggtaaagcgtgggggttgagttctacgcctgggaatgtgatggatatttttggtccttcttttaacgctattaatgagatgattaaaaacgctcaaacagccctagcaaaaacccaacagcttaacgctaatgaaaacgcccaaatcacgcaacccaacaatttcaacccctacacctctaaagacaaagggttcgctcaagaaatgctcaatagagctgaagctcaagcagagattttaaatttagctaagcaagtagcgaacaatttccacagcattcaagggcctattcaaggggatttagaagaatgtaaagcaggatcggctggcgtgatcactaataacacttggggttcaggttgcgcgtttgtgaaagaaactttaaactctttagagcaacacaccgcttattacggcaaccaggtcaatcaggatagggctttggctcaaaccattttgaattttaaagaagcccttaacaccctgaataaagactcaaaagcgatcaatagcggtatctccaacttgcctaacgctaaatctcttcaaaacatgacgcatgccactcaaaaccctaattccccagaaggtctgctcacttattctttggattcaagcaaatacaaccagctccaaaccatcgcgcaagaattgggcaaaaaccctttcaggcgctttggcgtgattgactttcaaaacaacaacggcgcaatgaacgggatcggcgtgcaagtgggttataaacaattctttggtaaaaaaaggaattgggggttaaggtattatggtttctttgattataaccatgcttatatcaaatctaattttttcaactccgcttctgatgtgtggacttatggggtgggtatggacgctctctataacttcatcaacgataaaaacaccaactttttaggcaagaacaacaagctttcagtagggctttttggaggctttgcgttagccgggacttcgtggcttaattcccaacaagtgaatttgaccatgatgaatggcatttataacgctaatgtcagcacttctaacttccaatttttgtttgatttaggcttgagaatgaacctcgctaggcctaagaaaaaagacagcgatcatgccgctcagcatggcattgaactaggttttaagatccccacgatcaacaccaactattattctttcatgggcgctaaactagaatacagaaggatgtatagcctttttctcaattatgtgtttgcttactaaaaattctttttgaacccctctttttttgggggagtgttgcaaaaatgcccccctatttgcttgtgagttttggttaaaattttagttacccacgcttaaaaagcgccaagccttttacacacaactcctttaattttgtttttaagaaa

SEQ ID NO: 12 - protein, 691 aa
  Signal Sequence 1...18
  fragment type: internal
MKKSLLLSLSLIASLSRAEDDGFYTSVGYQIGEAVQQVKNTGALQNLADRYDNLNNLLNQYNYLNSLVNLASTPSAITGAIDNLSSSAINLTSATTTSPAYQAVALALNAAVGMWQVIALFIGCGPGPTNNQSYQSFGNTPALNGTTTTCNQAYGTGPNGILSIDEYQKLNQAYQIIQTALNQNQGGGMPALNDTTKTGVVNIQQTNYRTTTQNNIIEHYYTENGKEIPVSYSGGSSFSPTIQLTYHNNAENLLQQAATIMQVLITQKPHVQTSNGGKAWGLSSTPGNVMDIFGPSFNAINEMIKNAQTALAKTQQLNANENAQITQPNNFNPYTSKDKGFAQEMLNRAEAQAEILNLAKQVANNFHSIQGPIQGDLEECKAGSAGVITNNTWGSGCAFVKETLNSLEQHTAYYGNQVNQDRALAQTILNFKEALNTLNKDSKAINSGISNLPNAKSLQNMTHATQNPNSPEGLLTYSLDSSKYNQLQTIAQELGKNPFRRFGVIDFQNNNGAMNGIGVQVGYKQFFGKKRNWGLRYYGFFDYNHAYIKSNFFNSASDVWTYGVGMDALYNFINDKNTNFLGKNNKLSVGLFGGFALAGTSWLNSQQVNLTMMNGIYNANVSTSNFQFLFDLGLRMNLARPKKKDSDHAAQHGIELGFKIPTINTNYYSFMGAKLEYRRMYSLFLNYVFAY

SEQ ID NO: 13 - DNA (Genomic DNA), 2270 bp; single-stranded, linear
  Coding Sequence 130...2049: (A) NAME/KEY Signal Sequence (B) LOCATION 130...193 (D) OTHER INFORMATION
attgagcgcatcaaaacaccctaaaacttttttgaaatccaataaatttatgttataattaaacgcattgtaaataaattctcattttgatacatttttacaataaaacattactttaaggaacatcttatgaaaaaaacgaaaaaaacgattctgctttctctaactctcgcggcgtcattgctccatgctgaagacaacggcgtttttttaagcgtgggttatcaaatcggtgaagcggttcaaaaagtgaaaaacgccgacaaggtgcaaaaactttcagacacttatgaacaattaagccggcttttaaccaacgataatggcacaaactcaaagacaagcgcgcaaatcaaccaagcggttaataatttgaacgaacgcgcaaaaactttagccggtgggacaaccaattcccctgcctatcaagccacgcttttagcgttgagatcggtgttagggctatggaatagcatgggttatgcggtcatatgcggaggttataccaaaagtccaggcgaaaacaatcaaaaagatttccactacaccgatgagaatggcaatggcactacaatcaattgcggtgggagcacaaatagtaatggcactcatagttctagtggcacaaatacattaaaagcagacaaaaatgtttctctatctattgagcaatatgaaaaaatccatgaagcttatcagattctttcaaaagctttaaaacaagccgggcttgctcctttaaatagcaaaggggaaaagttagaagcgcatgtaaccacatcaaaaccagaaaataatagtcaaactaaaacgacaacttctgttattgatacgactaatgatgcgcaaaatcttttgactcaagcgcaaacgattgtcaatacccttaaagattattgccccatgttgatagcgaaatctagtagtgaaagtagtggcgcagctactacaaacgccccttcatggcaaacagccggtggcggcaaaaattcatgtgcgacttttggtgcggagtttagtgccgcttcagacatgattaataatgcgcaaaaaatcgttcaagaaacccaacaactcagcgccaaccaaccaaaaaatatcacacaaccccataatctcaaccttaacacccctagcagtcttacggctttagctcaaaaaatgctcaaaaatgcgcaatctcaagcagaaattttaaaactagccaatcaagtggagagcgattttaacaaactttcttcaggccatcttaaagactacatagggaaatgcgatgcgagcgctataagcagtgcgaatatgacaatgcaaaatcaaaagaacaattgggggaacgggtgtgctggcgtggaagaaactctgtcttcattaaaaacaagtgccgctgattttaacaaccaaacgccacaaatcaatcaagcgcaaaacctagccaacacccttattcaagaacttggcaacaacccttttaggaatatgggcatgatcgcttcttcaaccacgaataacggcgccttgaatggccttggggtgcaagtgggttataagcaattttttggggaaaagaaaagatgggggttaaggtattatggtttctttgattacaaccacgcctatatcaaatccaatttctttaactcggcttctgatgtgtggacttatggggtgggcagcgatttattgtttaatttcatcaatgataaaaacaccaactttttaggcaagaataacaagatttcagtgggattttttggaggtatcgccttagcagggacttcatggcttaattctcaattcgtgaatttaaaaaccatcagcaatgtttatagcgctaaagtgaatacggctaacttccaatttttattcaatttgggcttgagaaccaatctcgctagacctaagaaaaaagatagtcatcatgcggctcaacatggcatggaattgggcgtgaaaatccctaccattaacacgaattattattcttttctagacactaaactagaatatcgaaggctttatagcgtgtatctcaattatgtgtttgcctattaaaaaccctctttttaaaaaagggggggctttaaaaaacctctaaagataaaaattttcaaaaaacaatcattaaaccctaaaaaagaaattttaaggtataatgctttcgccatttttaattttccatggcaaactcctttttagaatttatccccataatcgctcttatggggcgtttgttttgcaacaatcttttcgaaactatccaacaagcttta

SEQ ID NO: 14 - protein, 640 aa
  Signal Sequence 1...21
  fragment type: internal
MKKTKKTILLSLTLAASLLHAEDNGVFLSVGYQIGEAVQKVKNADKVQKLSDTYEQLSRLLTNDNGTNSKTSAQINQAVNNLNERAKTLAGGTTNSPAYQATLLALRSVLGLWNSMGYAVICGGYTKSPGENNQKDFHYTDENGNGTTINCGGSTNSNGTHSSSGTNTLKADKNVSLSIEQYEKIHEAYQILSKALKQAGLAPLNSKGEKLEAHVTTSKPENNSQTKTTTSVIDTTNDAQNLLTQAQTIVNTLKDYCPMLIAKSSSESSGAATTNAPSWQTAGGGKNSCATFGAEFSAASDMINNAQKIVQETQQLSANQPKNITQPHNLNLNTPSSLTALAQKMLKNAQSQAEILKLANQVESDFNKLSSGHLKDYIGKCDASAISSANMTMQNQKNNWGNGCAGVEETLSSLKTSAADFNNQTPQINQAQNLANTLIQELGNNPFRNMGMIASSTTNNGALNGLGVQVGYKQFFGEKKRWGLRYYGFFDYNHAYIKSNFFNSASDVWTYGVGSDLLFNFINDKNTNFLGKNNKISVGFFGGIALAGTSWLNSQFVNLKTISNVYSAKVNTANFQFLFNLGLRTNLARPKKKDSHHAAQHGMELGVKIPTINTNYYSFLDTKLEYRRLYSVYLNYVFAY

SEQ ID NO: 15 - DNA (Genomic DNA), 2248 bp; single-stranded, linear
  Coding Sequence 173...2128: (A) NAME/KEY Signal Sequence (B) LOCATION 173...224 (D) OTHER INFORMATION
tggttttatcgttacaaaattcaacatttcaaagataaataagttaaaataccccaaaatcttttttttttttttgaaatccaatcaatttatagtaaaattaggttcattgtaaatatattatcacttcatgatattcttacaacaaaaacattactttaaggaacattttatgaaaaagacaattctgctctctctctctgcttcatcgctcttgcacgctgaagacaacggcttttttgtgagcgccggctatcaaatcggcgaagcggtgcaaatggtcaaaaacaccggtgaattgaaaaacttgaacgaaaaatacgagcaattaagccagtatttaaatcaagtggcttcgttgaagcaaagcattcaaaacgccaacaacattgagctggtcaatagctctttaaactatttaaaaagctttaccaacaacaactataacagcaccacccaatcgcccatctttaatgccgtgcaagccgttatcacttcggtattgggtttttggagtctttatgcggggaattacttcactttttttgtgggtaaaaaggtgggtgatagtgggcaacccgctagtgtccagggtaaccctccttttaaaacgattatagagaactgctcaggaattgaaaactgcgctatggatcaaaccacttatgataagatgaaaaaactcgctgaagacctccaagcggctcaaacaaactctgccactaaaggcaacaatctttgcgctttatccgggtgtgctgcaacagactcaacatcaaacccaccaaactcaaccgtgagcaacgctcttaatttggcgcaacagcttatggatttaatcgcaaacactaaaacggctatgatgtggaaaaatatcgtcatcagtggcgtttcaaacacatccggtgctatcacatccactaattacccaacgcaatacgcggtgtttaacaacattaaggcgatgatacccattttgcaacaagcggttacgctttctcaaagcaaccacaccctatctgctagcttgcaagctcaagccacaggatctcaaacaaaccctaaattcgctaaagacatctacactttcgctcaaaaccaaaagcaagtcatctcttacgctcaagacattttcaacctctttaattctatccctgcagagcagtataagtatctagagaaagcttacttgaaaatacccaatgcgggttcaacgcctactaacccttacagacaagtggtgaatttaaaccaagaagttcagacgattaaaaacaatgtgagttattatggtaaccgggtggatgcggctttaagcgtggctagagatgtttataacctaaaatccaatcaagcagaaatcgtaaccgcctataacgacgctaagactttgagcgaagagatttctaaactcccgcacaatcaagtcaatacaaaagacattgttacactaccttacgataaaaacgccccagcagcaggccaatccaactaccaaatcaacccagagcagcaatccaatcttaaccaagctttagcagcgatgagcaataacccctttaaaaaagtgggcatgatcagctctcaaaacaataacggcgctttgaacgggcttggcgtgcaagtgggttataagcaattctttggcgaaagcaaaagatgggggttaaggtattacggattctttgattacaaccacggctacatcaaatccagcttctttaactcttcttctgatatatggacttatggcggtgggagcgatttgttagtgaatattatcaacgatagcatcacaagaaagaacaacaagctctccgtgggtctttttggaggcatccaactagcagggactacatggcttaattctcaatacgtgaatttaaccgcgttcaataacccttacagcgcgaaagtcaatgctaccaatttccaattcttgttcaatctcggcttgaggacgaatctcgctacagctaggaaaaaagacagcgaacattccgcgcaacatggcattgaattgggtattaaaatccccaccattaccacgaattactattcttttctaggcactcaattgcaatacagaaggctctatagcgtgtatctcaattatgtgttcgcttactgagtgattcaagctctcttctttaagggggtttagaaaaatcgcaacgccaagctttttatcgttggtgataaaatctacaaaactaacggcgcgacaacaaaccctaacgctacgctc

SEQ ID NO: 16 - protein, 652 aa
  Signal Sequence 1...17
  fragment type: internal
MKKTILLSLSASSLLHAEDNGFFVSAGYQIGEAVQMVKNTGELKNLNEKYEQLSQYLNQVASLKQSIQNANNIELVNSSLNYLKSFTNNNYNSTTQSPIFNAVQAVITSVLGFWSLYAGNYFTFFVGKKVGDSGQPASVQGNPPFKTIIENCSGIENCAMDQTTYDKMKKLAEDLQAAQTNSATKGNNLCALSGCAATDSTSNPPNSTVSNALNLAQQLMDLIANTKTAMMWKNIVISGVSNTSGAITSTNYPTQYAVFNNIKAMIPILQQAVTLSQSNHTLSASLQAQATGSQTNPKFAKDIYTFAQNQKQVISYAQDIFNLFNSIPAEQYKYLEKAYLKIPNAGSTPTNPYRQVVNLNQEVQTIKNNVSYYGNRVDAALSVARDVYNLKSNQAEIVTAYNDAKTLSEEISKLPHNQVNTKDIVTLPYDKNAPAAGQSNYQINPEQQSNLNQALAAMSNNPFKKVGMISSQNNNGALNGLGVQVGYKQFFGESKRWGLRYYGFFDYNHGYIKSSFFNSSSDIWTYGGGSDLLVNIINDSITRKNNKLSVGLFGGIQLAGTTWLNSQYVNLTAFNNPYSAKVNATNFQFLFNLGLRTNLATARKKDSEHSAQHGIELGIKIPTITTNYYSFLGTQLQYRRLYSVYLNYVFAY

SEQ ID NO: 17 - DNA (Genomic DNA), 2161 bp; single-stranded, linear
  Coding Sequence 122...2056: (A) NAME/KEY Signal Sequence (B) LOCATION 122...179 (D) OTHER INFORMATION
caaaaatcttttttttttttttttgaaatccaataaatttatggtaaagttaaacatattgtaaataaattttaatttctattcatgtttacaataaaaaaattactttaaggaacattttatgaaaaagacaattctactctctctctctctctcgctttcatcgctcttgcacgctgaagacaacggcttttttgtgagcgccggctatcaaatcggcgaacgggtgcaaatggtcaaaaacaccggcgaattgaaaaacttgaacgaaaaatacgagcaattaagccaatctttagcccaactggcttcgttaaaaaaaagcattcaaacggcgaacaacattcaggctgtcaacaatgctttaagcgatttaaaaagctttgcgagtaacaaccacacaaacaaagaaacatcgcccatctacaacaccgcgcaagctgttatcacttcagtattggctttttggagtctttatgcagggaacgctaccagttttcatgtgaccggtttgaatgatggatctaatgctcctcttggaagaatccatcaagatgggaactgcacaggattacaacaatgttttatgaataaagaaacttatgataaaatgaaagcgcttgccgaaaatctccaaaaagctcaaggcaatctctgtgccttatcagaatgccctagcgatcaattaaatggaaacaatggaaacaaaacttccatgactaaagctcttgaaaccgcgcaacagcttatggatttaatcgcaaacactaaaacggctatgatgtggaaaaatatcgtcatcgcaggtgttacaaacagacccggtggtgctggcgctatcacatccactggtcctgtaaccgactatgcggtgtttaacaacattaaggcgatgatacccattttgcaacaagcggttacgctttctcaaagcaaccacaccctatctgctagcttgcaagctcaagccacaggatctcaaacaaaccctaaattcgctaaagacatctacactttcgctcaaaaccaaaagcaagtcatctcttacgctcaagacattttcaacctctttaattctatccctgcagagcagtataagtatctagagaaagcttacttgaaaatacccaatgcgggttcaacgcctactaacccttacagacaagtggtgaatttaaaccaagaagttcagacgattaaaaacaatgtgagttattatggtaaccgggtggatgcggctttaagcgtggctagagatgtttataacctaaaatccaatcaagcagaaatcgtaaccgcctataacgacgctaagactttgagcgaagagatttctaaactcccgcacaatcaagtcaatacaaaagacattgttacactaccttacgataaaaacgccccagcagcaggccaatccaactaccaaatcaacccagagcagcaatccaatcttaaccaagctttagcagcgatgagcaataacccctttaaaaaagtgggcatgatcagctctcaaaacaataacggcgctttgaacgggcttggcgtgcaagtgggttataagcaattctttggcgaaagcaaaagatgggggttaaggtattacggattctttgattacaaccacggctacatcaaatccagcttctttaactcttcttctgatatatggacttatggcggtgggagcgatttgttagtgaatattatcaacgatagcatcacaagaaagaacaacaagctctccgtgggtctttttggaggcatccaactagcagggactacatggcttaattctcaatacgtgaatttaaccgcgttcaataacccttacagcgcgaaagtcaatgctaccaatttccaattcttgttcaatctcggcttgaggacgaatctcgctacagctaggaaaaaagacagcgaacattccgcgcaacatggcattgaattgggtattaaaatccccaccattaccacgaattactattcttttctaggcactcaattgcaatacagaaggctctatagcgtgtatctcaattatgtgttcgcttattaaaaaatcttctttttaaaatagggggagcttcatcaaatctattttgatagttatcaatatttgatgaaaataaagtcaaaaacaaaataaaccaaatcaccc

SEQ ID NO: 18 - protein, 645 aa
  Signal Sequence 1...19
  fragment type: internal
MKKTILLSLSLSLSSLLHAEDNGFFVSAGYQIGERVQMVKNTGELKNLNEKYEQLSQSLAQLASLKKSIQTANNIQAVNNALSDLKSFASNNHTNKETSPIYNTAQAVITSVLAFWSLYAGNATSFHVTGLNDGSNAPLGRIHQDGNCTGLQQCFMNKETYDKMKALAENLQKAQGNLCALSECPSDQLNGNNGNKTSMTKALETAQQLMDLIANTKTAMMWKNIVIAGVTNRPGGAGAITSTGPVTDYAVFNNIKAMIPILQQAVTLSQSNHTLSASLQAQATGSQTNPKFAKDIYTFAQNQKQVISYAQDIFNLFNSIPAEQYKYLEKAYLKIPNAGSTPTNPYRQVVNLNQEVQTIKNNVSYYGNRVDAALSVARDVYNLKSNQAEIVTAYNDAKTLSEEISKLPHNQVNTKDIVTLPYDKNAPAAGQSNYQINPEQQSNLNQALAAMSNNPFKKVGMISSQNNNGALNGLGVQVGYKQFFGESKRWGLRYYGFFDYNHGYIKSSFFNSSSDIWTYGGGSDLLVNIINDSITRKNNKLSVGLFGGIQLAGTTWLNSQYVNLTAFNNPYSAKVNATNFQFLFNLGLRTNLATARKKDSEHSAQHGIELGIKIPTITTNYYSFLGTQLQYRRLYSVYLNYVFAY

SEQ ID NO: 19 - DNA (Genomic DNA), 1799 bp; single-stranded, linear
  Coding Sequence 185...1633: (A) NAME/KEY Signal Sequence (B) LOCATION 185...233 (D) OTHER INFORMATION
tactcaaaacatttttcactatcaaaaaccttttttttaaatccaaaaaaaaagcaaaatttcttaatttttgctcaattttattaaaaattcaataaatttatggcacaatttaaacttattgtaaataaagtttcaatttgatacgattttacaaacaaaacattactttaaggaacattttatgaaaaaaacgattttactttctcttatggtttcatcgctcctcgctgaaaatgacggcgtttttatgagcgtgggctatcaaatcggcgaagcggttcaacaagtgaaaaacaccggcgaaatccaaaaagtctccaacgcttacgaaaatttgaacaatcttttaacccgctataacgaactcaaacaaacggcctctaacaccaattcaagtaccgctcaagcgattgataatctaaaagagagcgctagccgattgaaaacgacccccaatagcgctaatcaagccgtgtcttcagcgctcagctctgcggtagccatgtggcaagtaatagtctctaatttagccaataactcgctacccactagtgaatacaacaaaatcaatgcgatttctcaatcgctccaaaacaccctagaaaataaaaacaatgatcttaaaattgaaaatgactacgaccatcttttaactcaagctagcaccattattaatacccttcaaagccaatgcccaggcatagacggaggcaatggcaaaccatggggcattaatgcaagcgggaacgcatgcaatatttttggcaacacctttaacgccatcactagcatgatagatagcgctaaaaaagccgccgcagatgcccgaagaactgccccagaaagtccaaaccaaccaagtgcgtttaacaacgctgatttcaataaaaaccttaatcaagtctcaagcgttattaatgacacgatctcttacctcaaaggggacaatttagcaaccatctacaacacccttcaaaaaacgcccgattctaaagggtttcaaagtttggtgagccgatctagctatagttattccctcaacgaaacccaatattctgaattccaaactaccaccaaagagtttggccataacccttttagaagcgtgggtttaatcaactctcaaagcaataacggagcgatgaatggcgtgggcgtgcaattaggctataagcaattctttgggaaaaataaattttttgggatccgttattatgccttttttgattacaaccatgcctatatcaaatccaactttttcaactccgcttccaatgttttcacttatggcgcaggcagtgatcttttattgaatttcatcaatggcggatccgataaaaaccgcaaagtctcttttggcatttttggaggcatcgctctagcaggcacgacatggcttaattcccaatttatgaatttaaaaaccaccaatagcgcctacagcgctaagatcaacaacaccaatttccaattcttattcaatactggtttaaggcttcaagggattcaccatggcgttgaattaggcgtgaaaatccccaccatcaacacgaattactattctttcatgggcgctaaattagcataccgaagactttatagcgtgtatttcaattatgttttggcctattgatattgaatcggttctcattactaatgaggacaaagccaaactttttggctctcaatgaataacggcatcattttacttgactttttacaaaaaacacactaaaatttctttttcttttttgagcgaaattccagattagctcagcggtagagtaggcggctgt

SEQ ID NO: 20 - protein, 483 aa
  Signal Sequence 1...16
  fragment type: internal
MKKTILLSLMVSSLLAENDGVFMSVGYQIGEAVQQVKNTGEIQKVSNAYENLNNLLTRYNELKQTASNTNSSTAQAIDNLKESASRLKTTPNSANQAVSSALSSAVAMWQVIVSNLANNSLPTSEYNKINAISQSLQNTLENKNNDLKIENDYDHLLTQASTIINTLQSQCPGIDGGNGKPWGINASGNACNIFGNTFNAITSMIDSAKKAAADARRTAPESPNQPSAFNNADFNKNLNQVSSVINDTISYLKGDNLATIYNTLQKTPDSKGFQSLVSRSSYSYSLNETQYSEFQTTTKEFGHNPFRSVGLINSQSNNGAMNGVGVQLGYKQFFGKNKFFGIRYYAFFDYNHAYIKSNFFNSASNVFTYGAGSDLLLNFINGGSDKNRKVSFGIFGGIALAGTTWLNSQFMNLKTTNSAYSAKINNTNFQFLFNTGLRLQGIHHGVELGVKIPTINTNYYSFMGAKLAYRRLYSVYFNYVLAY

SEQ ID NO: 21 - DNA (Genomic DNA), 2338 bp; single-stranded, linear
  Coding Sequence 146...2218: (A) NAME/KEY Signal Sequence (B) LOCATION 146...200 (D) OTHER INFORMATION
acttaaaattgttttttttttttttcaaaatataaattttaagccaaaaataagcattttatggtaaaatggcgaactttcataaacatgactattatgggaatgtcatgggaatgtgaagaaaaatctattaaaaggagaaaacatgaaaaaatccctcttactctctctttctctcatcgcttccttatcaagagctgaagatgacggattttatacgagtgtgggctatcagatcggtgaagcggtccaacaagtgaaaaacacaggagcattgcaaaatcttgcagacagatacgataacttaaacaaccttttaaaccaatacaattatttaaattccttagtcaatttagccagcacgccgagcgcgatcaccggtgcgattgataatttaagctcaagcgcgattaacctcactagcgccaccaccacttcccccgcctatcaagctgtggctttagcgctcaatgccgctgtgggcatgtggcaagtcatagccctttttattggctgtggccctggccctaccaataatcaaagctatcaatcgtttggtaacacaccagcccttaatgggaccaccaccacttgcaatcaagcatatgggacaggccctaatggcatcctatctattgatgaataccaaaaactcaaccaagcttatcagatcatccaaaccgctttaaaccaaaatcaagggggtgggatgcctgccttgaatgacaccaccaaaacaggggtagtcaacatacaacaaaccaattataggaccaccacacaaaacaatatcatagagcattattatacagagaatgggaaagagatcccagtctcttattcaggcggatcatcattctcgcctacaatacaattgacataccataataacgctgaaaaccttttgcaacaagccgccactatcatgcaagtccttattactcaaaagccgcatgtgcaaacgagcaatggcggtaaagcgtgggggttgagttctacgcctgggaatgtgatggatatttttggtccttcttttaacgctattaatgagatgattaaaaacgctcaaacagccctagcaaaaacccaacagcttaacgctaatgaaaacgcccaaatcacgcaacccaacaatttcaacccctacacctctaaagacaaagggttcgctcaagaaatgctcaatagagctgaagctcaagcagagattttaaatttagctaagcaagtagcgaacaatttccacagcattcaagggcctattcaaggggatttagaagaatgtaaagcaggatcggctggcgtgatcactaataacacttggggttcaggttgcgcgtttgtgaaagaaactttaaactctttagagcaacacaccgcttattacggcaaccaggtcaatcaggatagggctttggctcaaaccattttgaattttaaagaagcccttaacaccctgaataaagactcaaaagcgatcaatagcggtatctccaacttgcctaacgctaaatctcttcaaaacatgacgcatgccactcaaaaccctaattccccagaaggtctgctcacttattctttggattcaagcaaatacaaccagctccaaaccatcgcgcaagaattgggcaaaaaccctttcaggcgctttggcgtgattgactttcaaaacaacaacggcgcaatgaacgggatcggcgtgcaagtgggttataaacaattctttggtaaaaaaaggaattgggggttaaggtattatggtttctttgattataaccatgcttatatcaaatctaattttttcaactccgcttctgatgtgtggacttatggggtgggtatggacgctctctataacttcatcaacgataaaaacaccaactttttaggcaagaacaacaagctttcagtagggctttttggaggctttgcgttagccgggacttcgtggcttaattcccaacaagtgaatttgaccatgatgaatggcatttataacgctaatgtcagcacttctaacttccaatttttgtttgatttaggcttgagaatgaacctcgctaggcctaagaaaaaagacagcgatcatgccgctcagcatggcattgaactaggttttaagatccccacgatcaacaccaactattattctttcatgggcgctaaactagaatacagaaggatgtatagcctttttctcaattatgtgtttgcttactaaaaactctctttaaaaaaggggtttgtttaaaaacgcttaaaagcatttttaaaattaagcagtaaagagcctagataatctcttgcaaccgctctcaagcgataaaattaaagtgat

SEQ ID NO: 22 - protein, 691 aa; identical to SEQ ID NO: 12
  Signal Sequence 1...18
  fragment type: internal

SEQ ID NO: 23 - DNA (Genomic DNA), 30 bp; single-stranded, linear
gccntcaaggagaaaacatgaaaaaaaccc

SEQ ID NO: 24 - DNA (Genomic DNA), 30 bp; single-stranded, linear
gccngaagacgacggcttttacacaagcgt

SEQ ID NO: 25 - DNA (Genomic DNA), 28 bp; single-stranded, linear
gccnaaagcttagtaagcgaacacataa

SEQ ID NO: 26 - DNA (Genomic DNA), 33 bp; single-stranded, linear
gccnaaggagaaaaaacatgaaaaaacacatcc

SEQ ID NO: 27 - DNA (Genomic DNA), 29 bp; single-stranded, linear
gccngaagacgacggcttttacacaagcg

SEQ ID NO: 28 - DNA (Genomic DNA), 30 bp; single-stranded, linear
gccnaacattagtaagcgaacacatagttc

SEQ ID NO: 29 - DNA (Genomic DNA), 33 bp; single-stranded, linear; identical to SEQ ID NO: 26

SEQ ID NO: 30 - DNA (Genomic DNA), 30 bp; single-stranded, linear; identical to SEQ ID NO: 24

SEQ ID NO: 31 - DNA (Genomic DNA), 27 bp; single-stranded, linear
gccnaaaagcttagtaagcgaacacat

SEQ ID NO: 32 - DNA (Genomic DNA), 30 bp; single-stranded, linear
gccnaaggagaaaacatgaagaaaaaattt

SEQ ID NO: 33 - DNA (Genomic DNA), 29 bp; single-stranded, linear
gccngaagacaacggcttttttgtgagtg

SEQ ID NO: 34 - DNA (Genomic DNA), 29 bp; single-stranded, linear
gccnagcttttagtaagcaaacacatagt

SEQ ID NO: 35 - DNA (Genomic DNA), 29 bp; single-stranded, linear
gccnaaggatatttatgaaaaaaaccctt

SEQ ID NO: 36 - DNA (Genomic DNA), 29 bp; single-stranded, linear
gccngaagacaacggcttttttatcagcg

SEQ ID NO: 37 - DNA (Genomic DNA), 30 bp; single-stranded, linear
gccngatattagtaagcaaacacataattc

SEQ ID NO: 38 - DNA (Genomic DNA), 31 bp; single-stranded, linear
gccnaaggagaaaacatgaaaaaatccctct

SEQ ID NO: 39 - DNA (Genomic DNA), 30 bp; single-stranded, linear
gccngaagatgacggattttatacgagtgt

SEQ ID NO: 40 - DNA (Genomic DNA), 30 bp; single-stranded, linear
gccnttttagtaagcaaacacataattgag

SEQ ID NO: 41 - DNA (Genomic DNA), 28 bp; single-stranded, linear
gccnaaggaacatcttatgaaaaaaacg

SEQ ID NO: 42 - DNA (Genomic DNA), 29 bp; single-stranded, linear
gccngaagacaacggcgtttttttaagcg

SEQ ID NO: 43 - DNA (Genomic DNA), 29 bp; single-stranded, linear
gccnggtttttaataggcaaacacataat

SEQ ID NO: 44 - DNA (Genomic DNA), 30 bp; single-stranded, linear
gccnaaggaacattttatgaaaaagacaat

SEQ ID NO: 45 - DNA (Genomic DNA), 29 bp; single-stranded, linear
gccngaagacaacggcttttttgtgagcg

SEQ ID NO: 46 - DNA (Genomic DNA), 27 bp; single-stranded, linear
gccntcactcagtaagcgaacacataa

SEQ ID NO: 47 - DNA (Genomic DNA), 29 bp; single-stranded, linear
gccnaaggaacattttatgaaaaagacaa

SEQ ID NO: 48 - DNA (Genomic DNA), 29 bp; single-stranded, linear
gccngaagacaacggcttttttgtgagcg

SEQ ID NO: 49 - DNA (Genomic DNA), 30 bp; single-stranded, linear
gccnttttaataagcgaacacataaaagag

SEQ ID NO: 50 - DNA (Genomic DNA), 30 bp; single-stranded, linear
gccnaaggaacattttatgaaaaaaacgat

SEQ ID NO: 51 - DNA (Genomic DNA), 29 bp; single-stranded, linear
gccngaaaatgacggcgtttttatgagcg

SEQ ID NO: 52 - DNA (Genomic DNA), 30 bp; single-stranded, linear
gccnatatcaataggccaaaacataattga

SEQ ID NO: 53 - DNA (Genomic DNA), 30 bp; single-stranded, linear
gccnaaggagaaaacatgaaaaaatccctc

SEQ ID NO: 54 - DNA (Genomic DNA), 30 bp; single-stranded, linear; identical to SEQ ID NO: 39

SEQ ID NO: 55 - DNA (Genomic DNA), 30 bp; single-stranded, linear; identical to SEQ ID NO: 40

SEQ ID NO: 56 - DNA, 32 bp; single-stranded, linear
cgcggatccgaatccaatttaatccaaaaagg

SEQ ID NO: 57 - DNA (cDNA), 33 bp; single-stranded, linear
ccgctcgagttaagtaagcgaacacatattcaa

SEQ ID NO: 58 - protein, 20 aa
EDDGFYTSVGYQIGEAAQMV

SEQ ID NO: 59 - DNA (cDNA), 31 bp; single-stranded, linear
ctgaattcgatttcaaggagaaaacatgaaa

SEQ ID NO: 60 - DNA (cDNA), 30 bp; single-stranded, linear
ccgctcgagttagtaagcgaacacataatt

SEQ ID NO: 61 - DNA (cDNA), 32 bp; single-stranded, linear; identical to SEQ ID NO: 56

SEQ ID NO: 62 - DNA (cDNA), 33 bp; single-stranded, linear
ccgctcgagttagtaagcgaacacatagttcaa

SEQ ID NO: 63 - DNA (cDNA), 29 bp; single-stranded, linear
cgcggatccgaagtttctttgtatcaaag

SEQ ID NO: 64 - DNA (cDNA), 33 bp; single-stranded, linear
ccgctcgagttagtaagcaaacacataattgtg